(19) United States
(12) Patent Application Publication
(10) Pub. No.: US 2025/0090311 A1
(43) Pub. Date: Mar. 20, 2025

(54) OUTER SKIRT FOR A PROSTHETIC HEART VALVE

(71) Applicant: EDWARDS LIFESCIENCES CORPORATION, Irvine, CA (US)

(72) Inventors: Nikolai Gurovich, Hadera (IL); Gil Senesh, Adi (IL); Michael Bukin, Pardes Hanna (IL); Anatoly Dvorsky, Haifa (IL); Tamir S. Levi, Zikhron Yaakov (IL); Elena Sherman, Pardes Hana (IL)

(21) Appl. No.: 18/950,681

(22) Filed: Nov. 18, 2024

Related U.S. Application Data
(63) Continuation of application No. PCT/US2023/025210, filed on Jun. 13, 2023.
(60) Provisional application No. 63/366,599, filed on Jun. 17, 2022.

(51) Int. Cl. A61F 2/24; A61F 2/00; A61L 27/18

(52) U.S. Cl. CPC A61F 2/2418; A61L 27/18; A61F 2002/0081; A61F 2210/0057; A61F 2210/0076; A61F 2220/0075; A61L 2430/20

(57) ABSTRACT

Outer skirts for a prosthetic heart valve are disclosed. As one example, a prosthetic heart valve can include an annular frame and an outer skirt disposed around an outer surface of the frame. The outer skirt can include a first portion comprising a polymeric material and a second portion comprising a fabric, where the polymeric material has a greater thromboresistance than the fabric. The second portion is secured to an inflow end of the frame and extends toward an intermediate portion of the frame and the first portion extends from the second portion and toward an outflow end of the frame

### CROSS-REFERENCE TO RELATED APPLICATIONS

This application is a continuation of PCT Patent Application No. PCT/US2023/025210 filed on Jun. 13, 2023, which application claims the benefit of U.S. Provisional Patent Application No. 63/366,599, filed Jun. 17, 2022, each of these applications being incorporated by reference herein in its entirety.

### FIELD

The present disclosure relates to prosthetic heart valves, and in particular to outer coverings or skirts comprising multiple layers for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, prosthetic heart valves comprise a cylindrical metal frame or stent and prosthetic leaflets mounted inside the frame. These valves can also include one or more coverings (or skirts) spanning a circumference of the frame, on an inner or outer surface of the frame. These coverings can be configured to establish a seal with the native tissue when the prosthetic heart valve is placed at the implantation site (and thus may be referred to as sealing members). However, the native tissue (e.g., at the native valve annulus or arterial wall around the native valve) can have an irregular shape while the frame of the prosthetic heart valve is generally cylindrical. As a result, gaps can be formed between the prosthetic heart valve and native heart valve annulus when the prosthetic heart valve is implanted within the native heart valve annulus, even when coverings are included on the prosthetic heart valve. Accordingly, a need exists for improved coverings or outer skirts for prosthetic heart valves.

### SUMMARY

Described herein are prosthetic heart valves, delivery apparatus, and methods for implanting prosthetic heart valves. In particular, described herein are examples of outer skirts for prosthetic heart valves and methods of making and using such outer skirts. Prosthetic heart valves can include a frame and a leaflet assembly arranged on an inner surface of the frame. The prosthetic heart valve can include an outer skirt arranged around a circumference of the frame and on an outer surface of the frame. The outer skirt can include two or more portions or layers comprising different materials. For example, an outer skirt can include a first portion comprising a polymeric material and a second portion comprising a fabric, the first portion extending further toward an outflow end of the frame than the second portion. In some examples, the first portion can be an inner layer of the outer skirt and the second portion can be an outer layer of the outer skirt. The first portion can have a higher thromboresistance than the second portion. As such, the skirts and prosthetic heart valves disclosed herein can, among other things, overcome one or more of the deficiencies of typical prosthetic heart valves.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, the sealing member can be an outer skirt disposed around an outer surface of the frame.

In some examples, the outer skirt can comprise a first portion comprising a polymeric material and a second portion comprising a fabric.

In some examples, the second portion is secured to an inflow end of the frame and extends toward an intermediate portion of the frame, and the first portion extends from the second portion and toward an outflow end of the frame.

In some examples, the outer skirt comprises an outer layer comprising a fabric and an inner layer comprising a polymeric material and covering an inner surface of the outer layer.

In some examples, the inner layer extends beyond an outflow edge portion of the outer layer toward an outflow end of the frame.

In some examples, the outer fabric layer is releasably attached to the inner layer.

In some examples, the sealing member comprises at least one layer comprising a plurality of micro scales formed on a surface thereof.

In some examples, the sealing member comprises a base layer comprising a polymeric material, the base layer disposed against an outer surface of the frame; and a plurality of outwardly extending yarns adhered to an outer surface of the base layer and extending radially outward and away from the base layer.

In some examples, a prosthetic heart valve comprises an annular frame having an inflow end, an outflow end, and an intermediate portion disposed between the inflow end and outflow end; and an outer skirt disposed around an outer surface of the frame. The outer skirt comprises a first portion comprising a polymeric material and a second portion comprising a fabric. The polymeric material has a greater thromboresistance than the fabric, the second portion is secured to the inflow end of the frame and extends toward the intermediate portion, and the first portion extends from the second portion and toward the outflow end of the frame.

In some examples, a prosthetic heart valve comprises an annular frame; and an outer skirt disposed around an outer surface of the frame. The outer skirt comprises an outer layer comprising a fabric and forming an exposed surface for contacting a tissue and an inner layer comprising a polymeric material and covering an inner surface of the outer layer, where the inner layer extends beyond an outflow edge portion of the outer layer toward an outflow end of the frame.

In some examples, a prosthetic heart valve comprises an annular frame having an inflow end, an outflow end, and an intermediate portion disposed between the inflow end and the outflow end; and an outer skirt disposed around an outer surface of the frame. The outer skirt comprises an inner layer comprising a polymeric material; and an outer layer comprising a fabric and attached to the inner layer. The outer layer comprises a first outer layer portion and a second outer layer portion, where the first outer layer portion extends from the inflow end of the frame toward the intermediate portion of the frame, and where the second outer layer portion extends from the first outer layer portion toward the outflow end. An outflow edge portion of the inner layer extends axially beyond an outflow edge portion of the second outer layer portion toward the outflow end of the frame such that the outflow edge portion of the inner layer is disposed closer to the outflow end of the frame than the outflow edge portion of the second outer layer portion.

In some examples, a prosthetic heart valve comprises an annular frame having an inflow end and an outflow end; and an outer skirt disposed around an outer surface of the frame. The outer skirt comprises an inner layer and a fabric outer layer releasably attached to the inner layer by a plurality of whip stitches and a pull suture. The plurality of whip stitches extends around the pull suture and through the inner layer and the outer layer, and the pull suture is configured to be pulled through and away from the whip stitches, thereby detaching the outer layer from the inner layer.

In some examples, a prosthetic heart valve comprises an annular frame; a valvular structure disposed in the frame and configured to regulate the flow of blood through the frame in one direction; and a skirt coupled to the frame, the skirt comprising at least one layer comprising a plurality of micro scales formed on a surface thereof.

In some examples, a prosthetic heart valve comprises an annular frame; and an outer skirt disposed around an outer surface of the frame. The outer skirt comprises a base layer comprising a polymeric material, the base layer disposed against the outer surface of the frame; and a plurality of outwardly extending yarns adhered to an outer surface of the base layer and extending radially outward and away from the base layer.

In some examples, a prosthetic heart valve comprises one or more of the components recited in Examples 1-78 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a side view of an example of a delivery apparatus configured to deliver and implant a radially expandable prosthetic heart valve at an implantation site.

FIG. 3 is a perspective view of a prosthetic heart valve including a fabric outer skirt disposed around an outer surface of a frame of the prosthetic heart valve.

FIG. 4 is a cross-sectional side view of the frame and outer skirt of the prosthetic heart valve of FIG. 3.

FIG. 5 is a cross-sectional side view of an exemplary prosthetic heart valve comprising an outer skirt with a polymeric inner layer and a fabric outer layer.

FIG. 6 is a cross-sectional side view of the prosthetic heart valve of FIG. 5 which illustrates an example where an outflow end portion of the inner layer of the outer skirt folds over an outflow end portion of the outer layer.

FIG. 7 is a cross-sectional side view of the prosthetic heart valve of FIG. 5 which illustrates an example where the outflow end portion and an inflow end portion of the inner layer of the outer skirt fold over the outflow end portion and the inflow end portion of the outer layer, respectively.

FIG. 8A is a cross-sectional side view of the prosthetic heart valve of FIG. 5 which illustrates an example where the outflow end portion and the inflow end portion of the inner layer of the outer skirt are folded over the outflow end portion and the inflow end portion of the outer layer, respectively, and fused together to form a loop at an outflow end portion and an inflow end portion of the outer skirt.

FIG. 8B is a cross-sectional side view of the prosthetic heart valve of FIG. 8A where the outer layer comprises a fabric base layer and a plurality of outwardly extending yarns.

FIG. 8C is a cross-sectional side view of the prosthetic heart valve of FIG. 8A where the outer layer comprises a plurality of outwardly extending yarns that are adhered directly to the inner layer.

FIG. 9 is a perspective view of another example of a prosthetic heart valve comprising a frame with a row of elongated cells at an outflow end of the frame and an outer skirt disposed around an outer surface of the frame and extending from inflow ends of the elongated cells to an inflow end of the frame.

FIG. 10 is a schematic of an exemplary skirt for a prosthetic heart valve in a flattened configuration, where the skirt comprises a polymeric first portion and a fabric second portion disposed adjacent one another.

FIG. 11 shows a portion of the outer skirt of FIG. 10 disposed around an outer surface of a portion of a frame of a prosthetic heart valve.

FIG. 12A is a cross-sectional side view of the outer skirt and frame of FIG. 11.

FIG. 12B is a cross-sectional side view of another exemplary outer skirt arranged on an outer surface of the frame of FIG. 11, where the polymeric first portion of the outer skirt forms an inner layer covering the fabric second portion of the outer skirt.

FIG. 13A is an SEM image of a shark skin surface comprising a riblet structure at a magnification of 50×.

FIG. 13B is an SEM image of the shark skin surface comprising the riblet structure at a magnification of 200×.

FIG. 13C is an SEM image of the shark skin surface comprising the riblet structure at a magnification of 1000×.

FIG. 14A is a schematic illustrating a method for replicating a shark skin surface by a PDMS elastomeric stamp method.

FIG. 14B is a schematic illustrating another method for replicating a shark skin surface by a PDMS embedded-elastomeric stamp method.

FIG. 15 is a cross-sectional side view of an outer skirt comprising simulated micro scales on a layer of the outer skirt and attached to a frame of a prosthetic heart valve.

FIG. 16 is a side view of a portion of another example of an outer skirt disposed around an outer surface of a portion of an exemplary frame of a prosthetic heart valve, the outer skirt comprising a polymeric inner layer and a fabric outer layer comprising two fabric outer layer portions, the inner layer extending beyond the fabric outer layer toward an outflow end of the frame.

FIG. 17 is a schematic cross-sectional side view of the outer skirt of FIG. 16 showing floating yarn sections of a first outer layer portion of the outer layer and a second outer layer portion of the outer layer extending from the first outer layer portion.

FIG. 18 is a schematic cross-sectional side view of the outer skirt of FIG. 16 disposed against an outer surface of the frame of FIG. 16, wherein an end portion of the second outer layer portion is wrapped around inflow apices of the frame.

FIG. 19 is a side view of a portion of another example of an outer skirt disposed around an outer surface of a portion of an exemplary frame of a prosthetic heart valve, the outer skirt comprising a polymeric inner layer and a fabric outer layer releasably attached to the inner layer by a pull suture and whip stitches, the inner layer extending beyond the fabric outer layer toward an outflow end of the frame.

FIG. 20 a schematic cross-sectional view of the outer skirt and frame of FIG. 19 showing the pull suture extending along an upper edge portion of the outer layer and whip stitches extending around the pull suture and through the outer skirt and the inner skirt.

FIG. 21 is a perspective view of the outer skirt of FIG. 19 disposed around an outer surface of the frame of a prosthetic heart valve.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

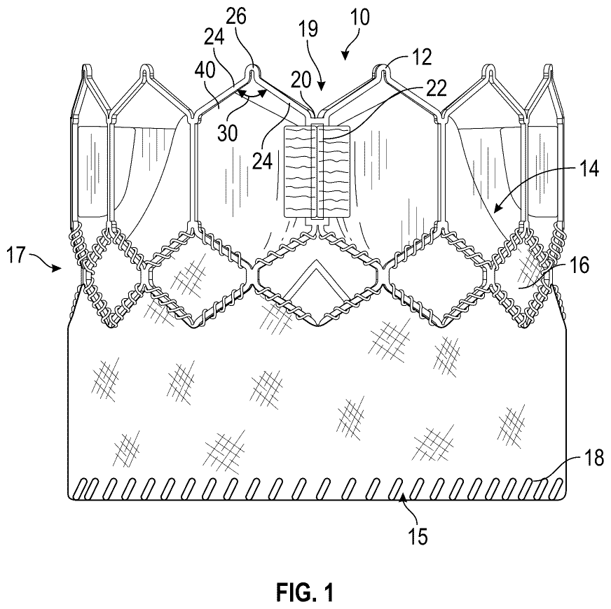

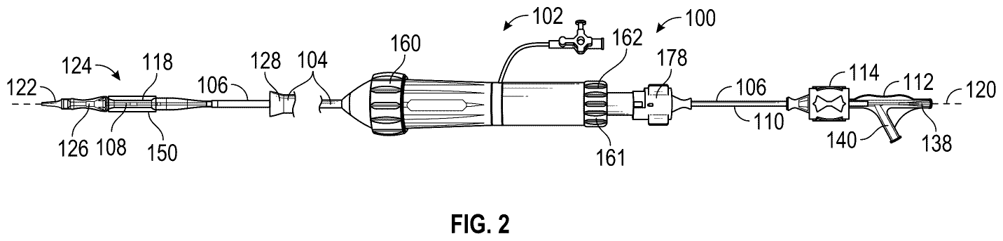

As introduced above, most prosthetic heart valve can include an outer skirt disposed around an outer surface of an annular frame of the prosthetic heart valve. The outer skirt can be configured to form a seal against native tissue upon implantation of the prosthetic heart valve, thereby reducing paravalvular leakage (PVL) past the prosthetic heart valve when expanded against the native anatomy. FIG. 1 illustrates an exemplary prosthetic device (e.g., prosthetic heart valve) comprising a frame, leaflets secured on an inside of the frame, and an outer skirt disposed around an outer surface of the frame. The prosthetic device can be advanced through a patient's vasculature, such as to a native heart valve, by a delivery apparatus, such as the exemplary delivery apparatus shown in FIG. 2.

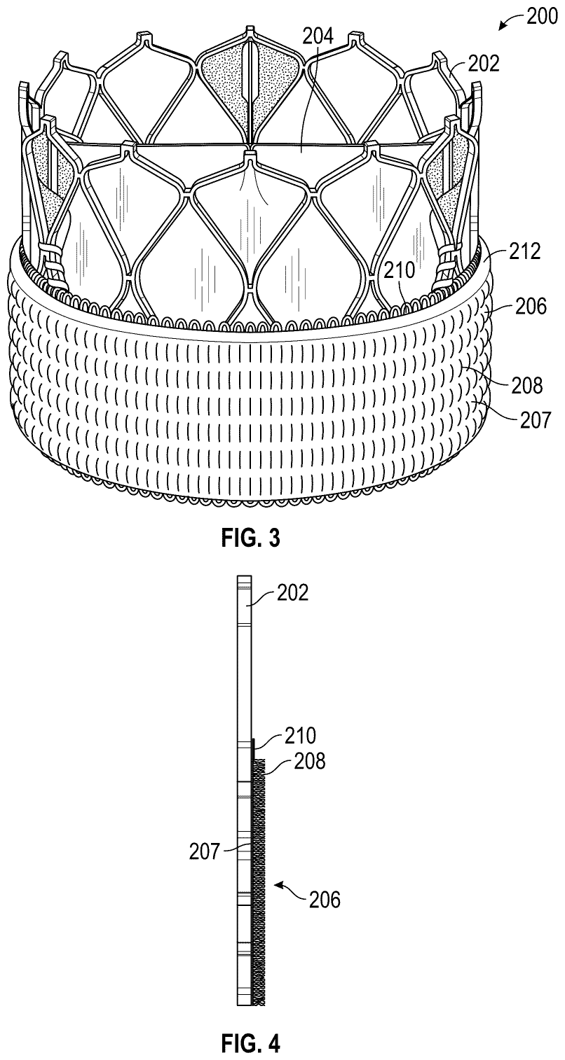

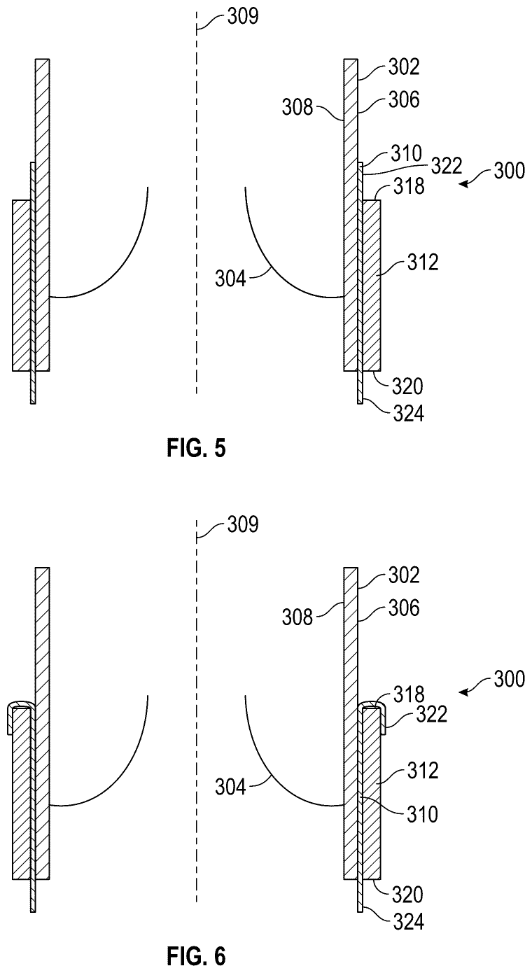

In some examples, an outer skirt for a prosthetic heart valve, such as the outer skirts depicted in FIGS. 3 and 4, can be configured with portions that extend radially outward from the frame of the prosthetic heart valve (toward the surrounding native anatomy) to increase PVL sealing against the native anatomy. For examples, the outer skirts shown in FIGS. 3 and 4 can comprise a relatively dense arrangement of outwardly protruding yarns. However, in some instances, such configurations can increase a crimp profile of the prosthetic heart valve when radially compressed onto a delivery apparatus. Thus, in some instances the outer skirt can comprise an inner thromboresistant layer and an outer fabric layer comprising a less dense arrangement of outwardly protruding yarns, as depicted in the various examples of FIGS. 5-8B. As a result, a density of the outwardly protruding yarns of the fabric layer can be reduced such that an overall crimp profile of the prosthetic heart valve is reduced, while still maintaining effective PVL sealing (via the thromboresistant inner layer).

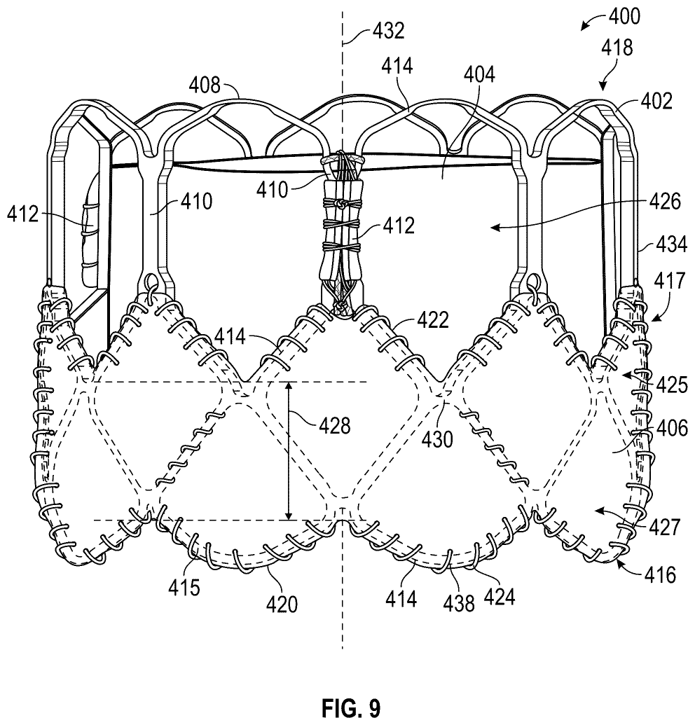

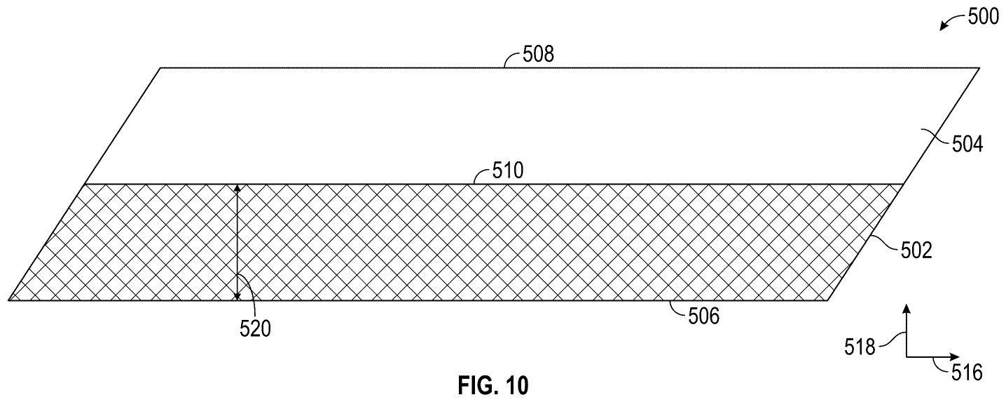

In some examples, the frame of the prosthetic heart valve can include a circumferentially extending first row of elongated first cells (at an outflow end of the frame). An outflow end of the outer skirt of the prosthetic heart valve can be secured to the frame along lower struts forming the first row of cells, as shown in FIG. 9. However, such a configuration can result in the outer skirt having a relatively short axial height, thereby leaving less surface area for sealing with the native anatomy at the implantation site. If the outflow end of the outer skirt were to be extended over a portion of the first row of cells (e.g., an inflow or lower end portion up the first cells), more surface area for PVL sealing would be created. However, due to the elongated axial length of the first cells, portions of the outer skirt extending across the first cells (between axial struts forming the upper cells) may extend or flutter into an interior of the prosthetic heart valve (through the first cells). Thus, in some instances, the outer skirt can comprise an outflow (or upper) portion comprising a polymeric material (e.g., thermoplastic polyurethane (TPU)) and an inflow (or lower) portion comprising a reinforced fabric (e.g., a woven fabric that encourages tissue ingrowth and PVL sealing against the native tissue), wherein the outflow portion is configured to stretch around and cover a portion of the first cells, as shown in FIGS. 10-12B. As a result, increased surface area for PVL sealing can be created without the outer skirt extending into an interior of the frame through the elongated first cells.

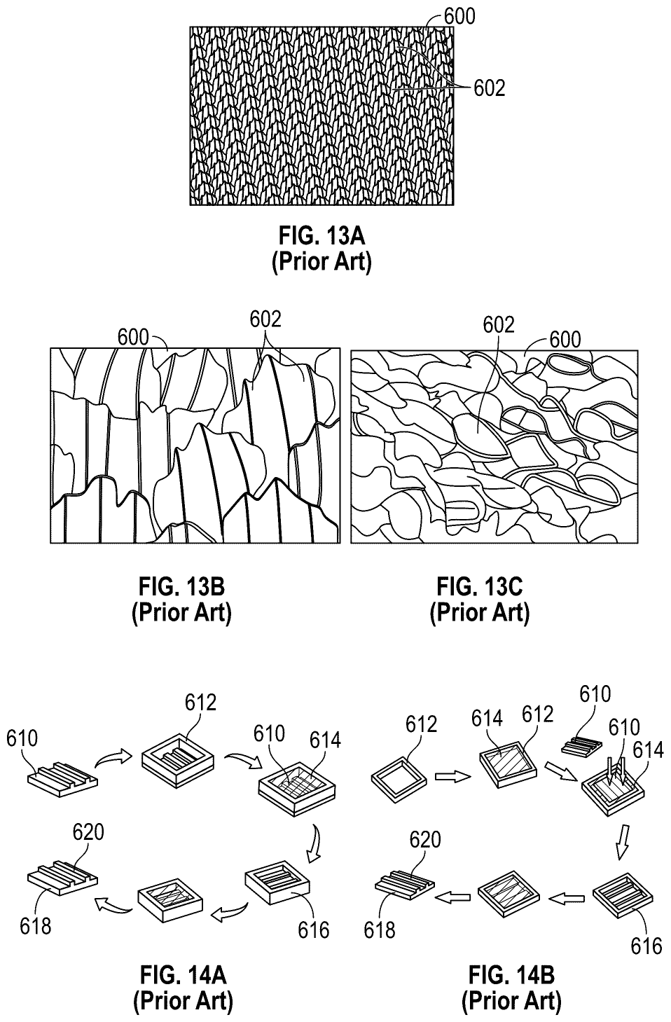

In some examples, a surface of a prosthetic heart valve, such as an inner surface of the outer skirt, can comprise a micro scale structure configured to reduce drag and that, in some instances, simulates shark scales. Examples of such surfaces and forming these surfaces are shown in FIGS. 13A-15. As a result, the inner surface of the outer skirt can be configured to reduce debris accumulation and/or calcification thereon, thereby increasing a long term durability of the outer skirt.

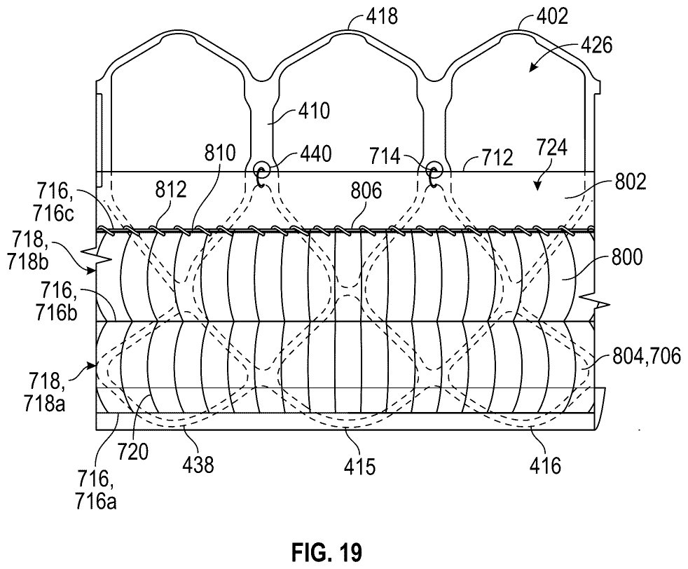

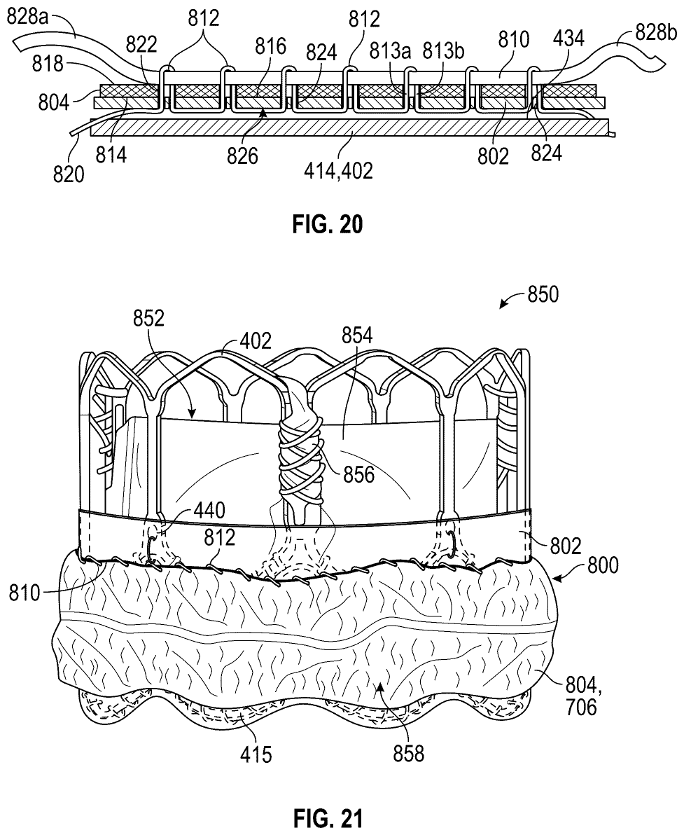

Further, in some examples, the outer skirt can comprise an inner layer comprising a polymeric material (such as TPU) and an outer layer attached to the inner layer and comprising one or more fabric portions that are configured to contact a tissue at the implantation site, as shown in FIGS. 16-21. An outflow edge portion of the inner layer can extend beyond an outflow edge portion of the outer layer. In some instances, the outer layer can comprise two fabric portions include a first outer layer portion comprising a fabric that wraps around inflow apices of the frame and a second outer layer portion comprising a plurality of floating fibers extending between leno lines of the second outer layer portion. The second outer layer portion can extend from the first outer layer portion toward the outflow end of the frame, where an outflow edge portion of the second outer layer portion is offset axially from the upper edge portion of the inner layer. In some instances, the outer layer can be releasably attached to the inner layer by a pull suture (FIGS. 19 and 20). As a result, the prosthetic heart valve, including the frame and inner layer of the outer skirt, can be more easily explanted during an explanation procedure.

### Examples of the Disclosed Technology

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 comprises four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12, while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other examples there can be greater or fewer number of leaflets (e.g., one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). In some examples, the leaflets 40 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

The frame 12 can be radially compressible (collapsible) and expandable (e.g., expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (e.g., stainless steel, etc.) or self-expanding materials (e.g., Nitinol). When constructed of a plastically-expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details regarding the prosthetic valve 10 and its various components are described in WIPO Patent Application Publication No. WO 2018/222799, which is incorporated herein by reference.

FIG. 2 shows a delivery apparatus 100, according to an example, that can be used to implant an expandable prosthetic heart valve (e.g., the prosthetic heart valve 10 of FIG. 1 and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site). Further details on the delivery apparatus 100 can be found in PCT Application No. PCT/US2021/047056, which is incorporated by reference herein.

FIGS. 3 and 4 depict another exemplary outer skirt 206 for a prosthetic heart valve 200 that is configured to increase PVL leakage sealing with the native anatomy once the prosthetic heart valve 200 has been radially expanded within the native anatomy (such as at a native heart valve annulus). In the example of FIG. 3, the outer skirt 206 is shown disposed around the outer surface of a radially expandable and compressible annular frame 202 of the prosthetic heart valve, while a plurality of leaflets 204 are secured to and disposed on an inside of the frame 202. The frame 202 can be similar to the frame 12 of the prosthetic valve 10 of FIG. 1. In some instances, the outer skirt 206 can be used in lieu of the outer skirt 18 on prosthetic valve 10.

The outer skirt 206 can comprise a fabric body formed from a plurality of strands or yarns that are woven, knitted, or otherwise secured together. In some instances, the outer skirt 206 can comprise a woven or knitted fabric comprising a fabric base layer 207 and a plurality of pile yarns (e.g., loop yarn) or floating yarns 208 that extend radially outward and away from the frame 202 (FIGS. 3 and 4). For example, the base layer 207 can comprise warp and weft yarns knitted into a mesh-like structure and the pile yarns or floating yarns 208 can be knit or woven into and extend outward from the base layer 207.

The pile yarns or floating yarns 208 can give the outer skirt 206 a fuzzy, plush, or velour-like appearance. Further, the outwardly protruding floating yarns 208 can be relatively densely packed together (as shown in FIG. 4) and configured to increase PVL sealing with the native anatomy upon implantation of the prosthetic heart valve 200. However, the densely packed floating yarns 208 can increase a crimp profile of the prosthetic heart valve 200 when radially compressed onto a delivery apparatus (such as the delivery apparatus 100 of FIG. 2).

Further details on skirts for prosthetic heart valves that comprise a knitted or woven material and pile or floating yarns are disclosed in U.S. Patent Publication 2019/0374337 and International Patent Publication WO 2021/202636, which are incorporated by reference herein.

In some examples, as shown in FIGS. 3 and 4, the outer skirt 206 can further comprise a plurality of loops 210 extending along an upper or outflow edge portion 212 of the outer skirt 206. However, when the leaflets 204 are in an open state they can contact the loops 210, or the floating yarns 208 (e.g., when the loops 210 are not included). Such contact can, in some instances, cause unwanted abrasion or wear of the leaflets 204.

Thus, the inventors herein have recognized that it is desirable to provide a prosthetic heart valve (or alternate prosthetic device) with an outer skirt comprising an inner thromboresistant layer or coating (which can comprise a polymeric layer, such as TPU) and an outer fabric layer than can comprise a plurality of outwardly protruding yarns. A density of the outwardly protruding yarns can be reduced relative to the yarns 208 of the outer skirt 206 due to the presence of the inner TPU layer that is configured to reduce or prevent PVL leakage, thereby reducing a crimp profile of the prosthetic heart valve to which the outer skirt is mounted.

FIGS. 5-8C illustrate examples of such outer skirts disposed around an outer surface 306 of an exemplary frame 302 of a prosthetic heart valve (which can be similar to or the same as frame 202 of FIG. 3 or frame 12 of FIG. 1). As shown schematically in FIGS. 5-8C, leaflets 304 of the prosthetic heart valve can be disposed on an inner surface 308 (or inside) of the frame 302 and the frame 302 can have a central longitudinal axis 309. Although the frame 302 is shown schematically in FIGS. 5-8C, the frame can have any of various configurations, such as the frame 12 of FIG. 1, or any of various frames that are balloon-expandable, self-expandable or mechanically expandable. Also, the frame 302 can be part of a prosthetic heart valve comprising one or more of the components described above for prosthetic valve 10, including leaflets 40 and inner skirt 16. In some examples, the prosthetic heart valve depicted in FIGS. 5-8C can be the prosthetic valve 10, except that the outer skirt 18 is replaced with the outer skirt 300. Additional details on balloon expandable prosthetic valves can be found in U.S. Pat. No. 9,393,110, and U.S. Provisional Application Nos. 63/178,416, filed Apr. 22, 2021, 63/194,830, filed May 28, 2021, and 63/279,096, filed Nov. 13, 2021, all of which are incorporated by reference herein. Additional details on a mechanically expandable prosthetic valve can be found in International Application PCT/US2021/052745, filed Sep. 30, 2021, and additional details on a self-expanding prosthetic valve can be found in U.S. Pat. No. 8,652,202, all of which are incorporated by reference herein.

Turning first to FIG. 5, an outer skirt 300 can comprise an inner layer 310 disposed against the outer surface 306 of the frame 302 and an outer layer 312 attached to the inner layer 310 and separated from the frame 302 by the inner layer 310. In some examples, the inner layer 310 can be disposed flush against the outer surface 306 of the frame 302. Further, in some instances, the outer layer 312 can be adhered or bonded directly to the inner layer 310, such as via an adhesive or via chemical bonding (e.g., heating). As explained further below, FIGS. 5-8B show various arrangements for end portions of the inner layer 310 relative to the outer layer 312.

The inner layer 310 can comprise a polymeric material. In some instances, the polymeric material can be relatively thromboresistant or have a thromboresistance that is greater than that of a fabric. Further, the polymeric material can be relatively non-abrasive or smooth. For example, in some instances, the polymeric material of the inner layer 310 can be TPU. In other instances, the inner layer 310 can comprise a different relatively thromboresistant material such as polytetrafluoroethylene (PTFE). In this way, tissue ingrowth may not occur on the inner layer 310 and contact between the leaflets 304 and the inner layer 310 may not result in abrasion of the leaflets 304. The inner layer 310 can be a relatively thin layer with a thickness in a range of 10-20 μm.

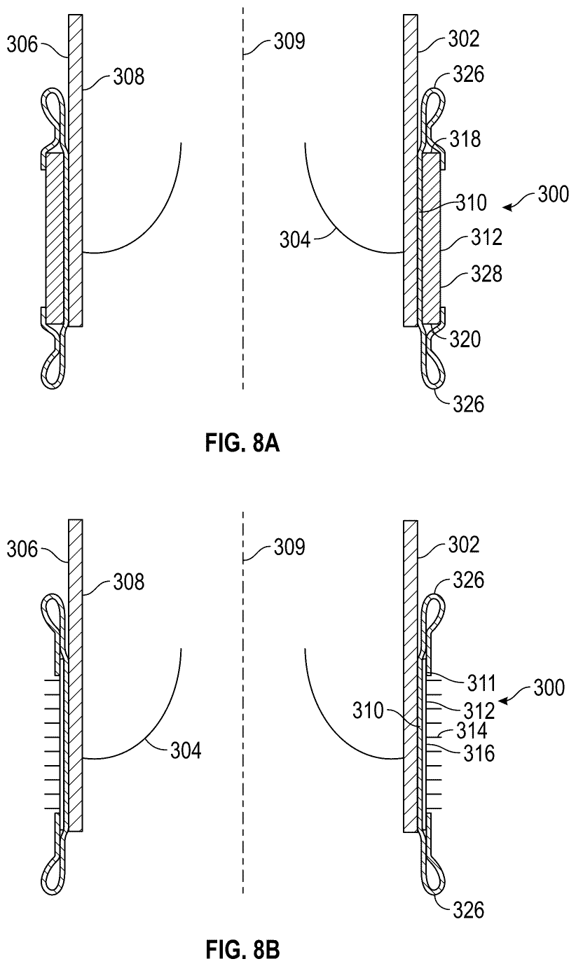

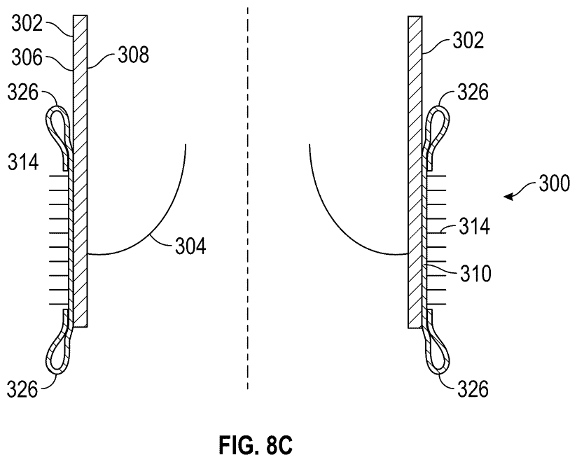

The outer layer 312 can be a fabric layer comprising a plurality of outwardly extending yarns 314, as depicted in FIG. 8B. For example, the outwardly extending yarns 314 can extend radially outward and away from the inner layer 310 and the frame 302.

In some instances, the outwardly extending yarns 314 can be knitted or woven into and extend outward from a base layer 316 of the outer layer 312 which can comprise a weave of one or more fibers or yarns. For example, in some instances, the outer layer 312 can be similar to the outer skirt 206 described above. In some instances, the outer layer 312 can comprise circumferentially extending rows of a woven portions with the outwardly extending yarns 314 extending between adjacent woven portions. For such outer layers 312, a density of the outwardly extending yarns 314 can be reduced compared to the outer skirt 206 of FIG. 4 due to the inclusion of the inner layer 310 (which can prevent leakage through the outer skirt 300).

In other instances, the outwardly extending yarns 314 of the outer layer 312 can be individual textured yarns 314 that are adhered directly to the inner layer 310 (e.g., without a fabric base layer 316 therebetween). For example, as depicted in FIG. 8C, an outer skirt 350 can comprise the inner layer 310 and an outer layer comprising the plurality of outwardly extending yarns 314 adhered or bonded directly to the inner layer 310 (e.g., via an adhesive or chemical bonding, such as via heating or another bonding method). The outwardly extending yarns 314 can be spaced apart from one another across the outer surface of the inner layer 310. Since the outwardly extending yarns 314 can be individually bonded directly to the polymeric inner layer 310, instead of being woven or knitted into a fabric structure or base layer, a density or number or outwardly extending yarns 314 can be further reduced and selected to achieve a desired crimp profiled for a selected prosthetic heart valve, while still providing adequate sealing with the native tissue (upon implantation of the prosthetic heart valve). As a result, the outer layer comprising the outwardly extending yarns 314 can be more easily customized for a selected prosthetic heart valve and/or application.

In some examples, the outer layer 312 can comprise a plurality of loops (one loop 311 shown in the cross-section of FIG. 8B) extending along an upper edge 318 (or outflow edge) of the outer layer 312 (e.g., similar to the loops 210 shown in FIG. 3). In such examples, an upper edge portion 322 (or outflow edge portion) of the inner layer 310 can extend beyond (above in FIG. 5) the upper edge 318 of the outer layer 312 (FIG. 5). In some instances, the upper edge portion 322 can extend far enough beyond the upper edge 318 (in the axial direction) such that the upper edge portion 322 of the inner layer 310 covers and blocks the loops 311 of the upper edge 318 of the outer layer 312, and/or the outwardly extending yarns 314, from contacting the leaflets 304.

In some instances, as shown in FIG. 6, the upper edge portion 322 of the inner layer 310 can wrap around the upper edge 318 of the outer layer 312, thereby forming a “J” shape with the upper edge portion 322. As such, the upper edge portion 322 of the inner layer 310 can extend over, and in some instances cover, the loops 311 extending from the upper edge 318 of the outer layer 312 (as shown in FIG. 8B).

In some examples, as shown in FIGS. 5 and 6, a lower edge portion 324 (or inflow edge portion) of the inner layer 310 can extend beyond (below in FIGS. 5 and 6) a lower edge 320 (or inflow edge) of the outer layer 312. In some instances, the lower edge portion 324 can extend beyond the lower edge 320, and beyond an inflow end of the frame 302, by an amount that is one quarter to one third of an outer diameter of the frame 302.

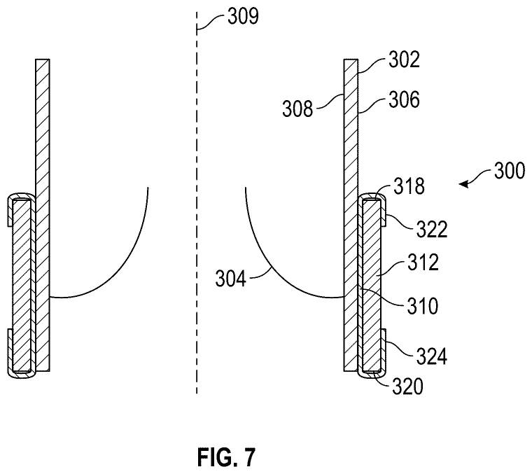

In some instances, as shown in FIG. 7, the lower edge portion 324 of the inner layer 310 can wrap around the lower edge 320 of the outer layer 312, thereby forming a “J” shape with the lower edge portion 324. As such, the lower edge portion 324 can extend over the lower edge 320 and cover a portion of an outer surface of a lower edge portion of the outer layer 312.

By extending the lower edge portion 324 of the inner layer 310 beyond (FIGS. 5 and 6) and/or wrapping it around the lower edge 320 and over a portion of an outer surface of the outer layer 312 (FIG. 7), tissue ingrowth in this region of the outer skirt 300 can be reduced, thereby providing easier access to lower portions of the prosthetic heart valve (e.g., at or below the native valve annulus) during an explanation procedure. As a result, explanation of the prosthetic heart valve from the native valve annulus can be made easier.

In some examples, as shown in FIGS. 8A and 8B, the upper edge portion 322 and/or the lower edge portion 324 of the inner layer 310 can be folded over and fused so as to form a loop 326 extending above or below the respective upper edge 318 or lower edge 320 of the outer layer 312, while a remainder (e.g., slack or free end portion) of the folded upper edge portion 322 or lower edge portion 324 further extends over the respective upper edge 318 or lower edge 320. Further, in some instances, the remainder of the folded over upper edge portion 322 or lower edge portion 324 can extend along a portion of the outer surface 328 of the outer layer 312 such that the respective upper edge 318 or lower edge 320 is covered (FIGS. 8A-8B).

FIG. 9 depicts another exemplary prosthetic heart valve 400 comprising a radially expandable and/or compressible annular frame 402, a plurality of leaflets 404 mounted within the frame 402, and an outer skirt 406 secured to and around an outer surface 434 of the frame 402. The frame 402 can comprises a plurality of interconnected struts 414 and a plurality of apices (outflow apices 408 and inflow apices 438) that are spaced circumferentially apart around an inflow end 416 (or “inflow end portion”) and an outflow end 418 (or “outflow end portion”) of the frame 402. Each apex 408, 438 is formed at a junction between two angled struts 414 at either the inflow end 416 or the outflow end 418. The frame 402 comprises a plurality of axially extending struts 410 (referred to herein as “axial struts 410”), some of which define commissure windows therein. Commissure tabs of adjacent leaflets 404 can be paired together and extend through the commissure windows, thereby forming commissures 412 secured to the frame 402. The axial struts 410, together with a first row of struts 414 forming the outflow end 418 and a second row of struts disposed adjacent to the first row of struts (in the axial direction), can form a circumferentially extending row of outflow cells 426 (which can also be referred to as “first cells” or “upper cells”). The outflow cells 426 can be elongated in the axial direction relative to cells in remaining rows of cells of the frame 402 (e.g., a second row of intermediate cells 425 disposed proximate an intermediate portion 417 of the frame 402 and a third row of inflow cells 427 disposed at the inflow end 416 of the frame 402). Additional details on the prosthetic heart valve 400, including details on the frame 402, can be found in U.S. Provisional Application No. 63/279,096, as already incorporated by reference above.

The outer skirt 406 of the prosthetic heart valve 400 can include an inflow edge portion 420 (or lower edge portion) that is secured to struts 414 forming the inflow end 416 of the frame 402 (inflow struts 415) via one or more fasteners (e.g., whip stitches 424 shown in FIG. 9). The outer skirt 406 can also include an outflow edge portion 422 (or upper edge portion) that is secured to struts 414 of the second row of struts that form lower or inflow edges of the outflow cells 426. In some instances, as shown in FIG. 9, the outflow edge portion 422 of the outer skirt 406 can extend along and follow a shape of the struts 414 forming the inflow edges of the outflow cells 426, thereby causing the outflow edge portion 422 to follow a zig-zag pattern of the struts 414 of the outflow cells 426. While such a configuration can result in a tight attachment of the outflow edge portion 422 of the outer skirt 406 to the frame 402, it can also result in the outer skirt 406 having a relatively short minor axial height 428 (e.g., the height along portions of the outer skirt 406 that extend axially from the non-apical junctions at the inflow end 416 to bottom junctions of the outflow cells 426) (FIG. 9).

The inventors herein have recognized that while it may be desirable to extend the outer skirt beyond the inflow struts 414 of the outflow cells 426 (for example, toward the outflow end 418 and closer to a mid-height of the outflow cells 426) in order to increase a surface area for PVL sealing at the implantation site, this may cause portions of the outer skirt 406 extending across the outflow cells 426 to extend into or loosely dangle across the outflow cells 426 (due to the relatively large size of the outflow cells 426).

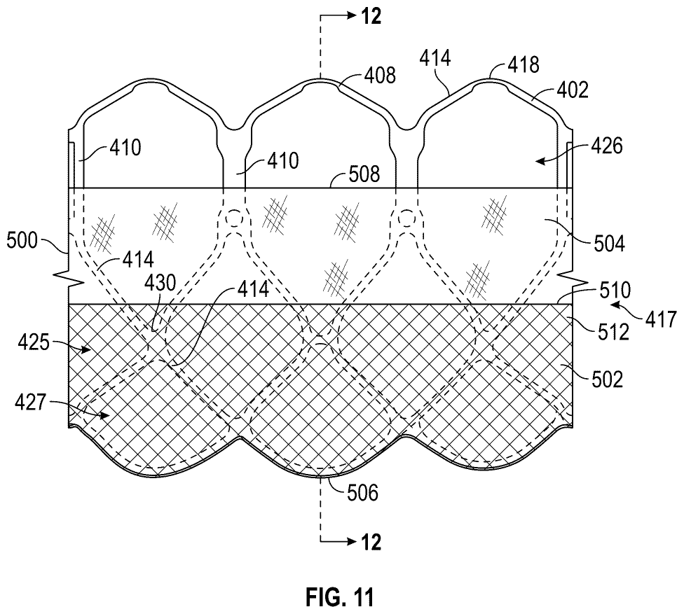

In some examples, as shown in FIGS. 10-12A, an outer skirt 500 for a prosthetic heart valve (such as the prosthetic heart valve 400 of FIG. 9) can comprise a first portion 502 comprising a first material (e.g., a fabric) and a second portion 504 comprising a second material (e.g., a polymeric material). The first portion 502 can define an inflow edge portion 506 of the outer skirt 500 that, in some instance, is configured to be secured to an inflow end of a frame of the prosthetic heart valve. The second portion 504 can define an outflow edge portion 508 of the outer skirt 500 that is configured to extend toward an outflow end of the frame and, in some instances, over a portion of elongated outflow cells of the frame. In some examples, instead of being relatively straight and parallel to the outflow edge portion 508, the inflow edge portion 506 of the outer skirt 500 can have an undulating shape that matches and follows a shape of the struts at the inflow end of the frame (e.g., as shown in FIG. 11).

Although the outer skirt 500 and additional outer skirts described herein (e.g., outer skirts 650, 700, and 800) are depicted as being secured to the frame 402, the frame can have any of various configurations, such as the frame 12 of FIG. 1, or any of various frames that are balloon-expandable, self-expandable or mechanically expandable, such as disclosed in the applications referenced above.

In some examples, as shown in FIG. 11, the inflow edge portion 506 of the outer skirt 500 can be secured to the struts 414 defining the inflow end 416 of the frame 402. The second portion 504 extends from an upper or outflow end of the first portion 502 (around the intermediate portion 417 of the frame 402), toward the outflow end 418, and over a portion of the elongated outflow cells 426 of the frame 402. In some instances, as shown in FIG. 11, the outflow edge portion 508, which is an upper or free edge of the second portion 504, can extend circumferentially along the frame 402 at a mid-portion (or mid-height in the axial direction) of the elongated outflow cells 426. For example, the outflow edge portion 508 can extend across the axial struts 410 at approximately a mid-height of the axial struts (e.g., between an inflow and outflow end of the axial struts 410). In some instances, the outflow edge portion 508 can be disposed closer to the outflow end 418 of the frame 402 than shown in FIG. 11, such that the second portion 504 extends across approximately ½ to ¾ the axial height of the outflow cells 426.

An interface 510 (or intersection) between the first portion 502 and the second portion 504 of the outer skirt 500 on an outer surface 512 (a radially outward facing surface that faces radially outward and away from the frame 402) of the outer skirt 500 can be disposed along the frame 402 proximate or adjacent to lower or inflow apices 430 of the outflow cells 426 (FIG. 11). In some instances, the interface 510 can be closer to the inflow end 416 than shown in FIG. 11, such that the first portion 502 only extends to the inflow apices 430 of the outflow cells 426. Said another way, the first portion 502 can have an axial height 520 (FIG. 10) that is specified such that the first portion 502 covers a portion of the frame 402 that is disposed between the inflow end 416 and the inflow apices 430 of the outflow cells 426.

In some examples, the first portion 502 and the second portion 504 can be secured together, along the interface 510, via one or more fasteners (e.g., stitches). In some instances, the first portion 502 and the second portion 504 can at least partially overlap one another along the interface 510 and can be secured together by an adhesive or bonding (e.g., chemical bonding).

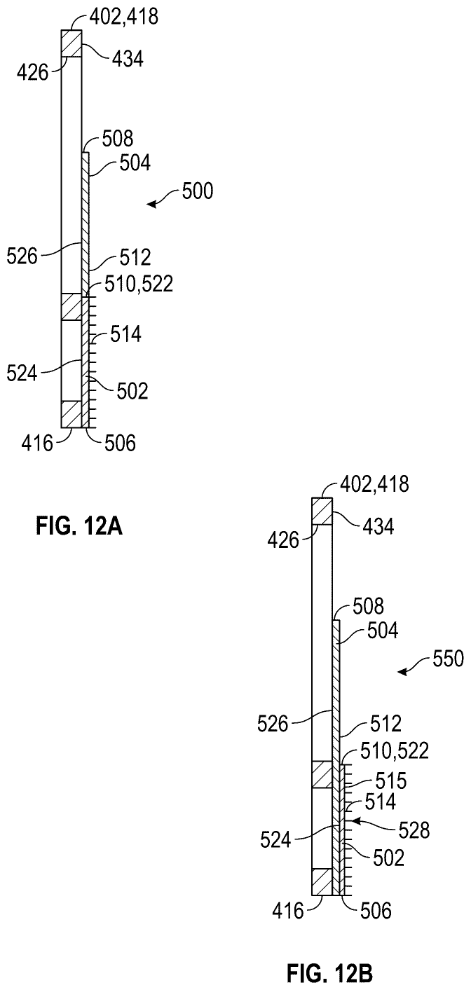

The first portion 502 can be a reinforced layer comprising a fabric (such as a woven fabric). In some instances, the first portion 502 can comprise a fabric (such as a polyethylene terephthalate, or PET, fabric) with outwardly extending fibers 514 (which, for example, can be the same or similar to the outwardly extending or protruding yarns 208 in FIG. 4 or 314 in FIGS. 8A and 8B) (FIG. 12A). In some instances, the outwardly extending fibers 514 can be leno yarns or fibers that are woven in a leno weave pattern with a base fabric layer 515 of the first portion 502, such as disclosed in U.S. Patent Publication 2019/0374337, as already incorporated by reference above. In some instances, the first portion 502 can comprise a fabric formed by fibers that are woven together and angled at approximately 45° with respect to a horizontal or circumferential direction (when disposed around the frame 402), as depicted schematically in FIGS. 10 and 11 (FIG. 10 shows reference axes illustrating the circumferential direction 516 and axial direction 518, which can be relative to a central longitudinal axis 432 of the frame 402 (FIG. 9) when the outer skirt 500 is disposed around the outer surface of the frame 402, as shown in FIG. 11). The angled fibers of the first portion 502 can provide the outer skirt 500 with increased longitudinal extendibility (in the axial direction 518), when the prosthetic heart valve is radially compressed into a compressed configuration (e.g., for delivery on a delivery apparatus, such as shown in FIG. 2).

The second portion 504 can be a polymeric layer comprising a polymeric material. In some examples, the polymeric material can have a thromboresistance that is greater than the fabric of the first portion 502. In some instances, the polymeric material is a non-textile. Further, in some instances, the polymeric material is elastic and is configured to stretch circumferentially when the frame 402 is radially expanded from a radially compressed configuration to a radially expanded configuration. As such, the second portion 504 can form snug fit around the frame 402 when the frame 402 is in a radially expanded configuration. As a result, the second portion 504 can extend along the outer surface 434 of the frame 402 without fluttering along or extending through the outflow cells 426 into the frame 402 (e.g., toward the leaflets 404). It should be noted that, as used herein, “elastic” or “elastic material” can refer to elastic and partially elastic materials (e.g., entering plasticity), as long as the material retains its structural integrity under stretching (e.g., does not tear).

In some examples, the polymeric material of the second portion 504 can be TPU. In alternate examples, the second portion 504 can comprise a different polymeric material, such as ePTFE. In some instances, the second portion 504 can be configured to stretch in both the axial direction 518 and the circumferential direction 516.

In some examples, as depicted in FIG. 12A, the first portion 502 and the second portion 504 can be disposed adjacent to one another (or connected to one another at the interface 510), such that the second portion 504 extends from an upper or outflow end 522 of the first portion 502. Thus, in some instances, the outer skirt 500 can comprise a single layer with the first portion 502 forming a lower portion of the outer skirt 500 and the second portion 504 forming an upper portion of the outer skirt 500. In such examples, both an inner surface 524 of the first portion 502 and an inner surface 526 of the second portion 504 can be disposed adjacent to an outer surface 434 of the frame 402, without any additional intervening layers therebetween. In some instances, at least the inner surface 526 of the second portion 504 can be flush against the outer surface 434 of the frame 402.

In alternative examples, as depicted in FIG. 12B, an outer skirt 550 can comprise the first portion 502 and the second portion 504, but the second portion 504 can extend along the inner surface 524 of the first portion 502 such that the second portion 504 covers all or a majority of the inner surface 524 of the first portion 502. As a result, the inner surface 526 of the second portion 504 can be disposed against the outer surface 434 of the frame 402 and disposed between (separating) the frame 402 and the first portion 502. Thus, in some instances, the outer skirt 550 can comprise 500 two layers with the second portion 504 forming a first, inner layer of the outer skirt 500 and the first portion 502 forming a second, outer layer of the outer skirt 550 which extends from the inflow edge portion 506 to a location at a middle portion of the outer skirt 550 that is between the inflow edge portion 506 and the outflow edge portion 508 (at the interface 510 in FIG. 12B). Said another way, the outer skirt 550 can comprise an outflow or upper portion comprising only the polymeric second portion 504 and an inflow or lower portion comprising the fabric first portion 502 (as an outer layer) and the polymeric second portion 504 (as an inner layer) (FIG. 12B).

In some instances, instead of the fibers 514 of the first portion 502 extending outward from a base fabric layer 515 of the first portion 502, the fibers 514 forming the first portion 502 can be at least partially embedded within and extend outward from a lower region of the second portion 504 (e.g., the region between the inflow edge portion 506 and the interface 510). For example, in some instances, the fibers 514 forming the second portion 502 can be adhered or bonded directly to the lower regions of the second portion 504, similar to as described above with reference to FIG. 8C.

In some instances, an outer surface 528 of the first portion 502 can also be covered by the second portion 504. For example, in some instances the first portion 502 can be encapsulated by and between layers of the second portion 504. However, the second portion 504 can still extend beyond the first portion 502 over a portion of the outflow cells 426, as described above, such that an upper or outflow portion of the outer skirt 500 only includes the second portion 504 and is devoid of the fabric or fibers of the first portion 502.

In this way, the second portion 504 can be configured to reduce or prevent PVL across the frame 402 (e.g., over upper regions of a scallop line of the leaflets), while the first portion 502 further serves to prevent PVL axially along the prosthetic heart valve, at portions between the frame 402 and the native tissue at the implantation site. Further, the first portion 502 can promote tissue ingrowth therein.

In some instances, since stretchable or elastic materials can creep over time, a prosthetic heart valve including the outer skirt 500 can be stored in a partially compressed state (rather than fully expanded), thereby preventing creeping and loss of tension in the second portion 504 prior to valve implantation. For example, the prosthetic heart valve can be radially expanded and then partially compressed to a diameter that is smaller than the fully expanded diameter and then stored in this state until it's ready for implantation.

In some examples, a component of a prosthetic heart valve, such as an inner skirt (e.g., inner skirt 16 of FIG. 1), an outer skirt (e.g., any one of the outer skirts 18 of FIG. 1, 206 of FIG. 3, 300 of FIGS. 5-8B, 406 of FIG. 9, 500 of FIGS. 10-12B, 700 of FIG. 16-18, or 800 of FIGS. 19-21), or an alternate skirt or connecting component can have a surface configured to mimic a structure of a shark skin. In this way, a drag of flow over the surface can be reduced. As a result, the skirt or connecting component of the prosthetic heart valve can be more resistant to calcification and/or debris accumulation when implanted in a patient. It should be noted that the micro scale structure described below can also be implemented on surfaces of additional implantable medical devices, such as stents.

FIGS. 13A-13B show SEM images of a shark skin surface 600 at magnifications of 50× (FIG. 13A), 200× (FIG. 13B), and 1000× (FIG. 13C). FIGS. 13A-13B show a riblet structure, or riblets 602, of the shark skin surface 600 (see, e.g., Xia et al. Using Bio-Replicated Forming Technologies to Fabricate Shark-Skin Surface. Braz. arch. biol. technol. 2015; 60). The riblets 602 of the shark skin surface 600 can reduce drag of flow over the shark skin surface 600.

A shark skin surface, such as the shark skin surface 600 of FIGS. 13A-13C can be replicated (at a micro scale) by a PDMS elastomeric stamp method (as shown in FIG. 14A) or by a PDMS embedded-elastomeric stamp method (FIG. 14B) (see, e.g., Xia et al. Using Bio-Replicated Forming Technologies to Fabricate Shark-Skin Surface. Braz. arch. biol. technol. 2015; 60). As shown in FIGS. 14A and 14B, a shark skin sample 610 can be inserted into a plate 612 that is filled with PDMS 614 (FIG. 14A) or pressed into a plate 612 containing PDMS 614 (FIG. 14B) to form a negative mold 616 of the shark skin sample 610. The negative mold 616 can then be filled with a desired material, such as TPU, ePTFE, or the like to form a material layer 618 comprising simulated micro scales 620.

In some instances, the micro scale structure can be formed on a surface of a skirt of a prosthetic heart valve. In some examples, the surface can be an inner surface (e.g., the surface facing radially inward toward a central longitudinal axis of the prosthetic heart valve).

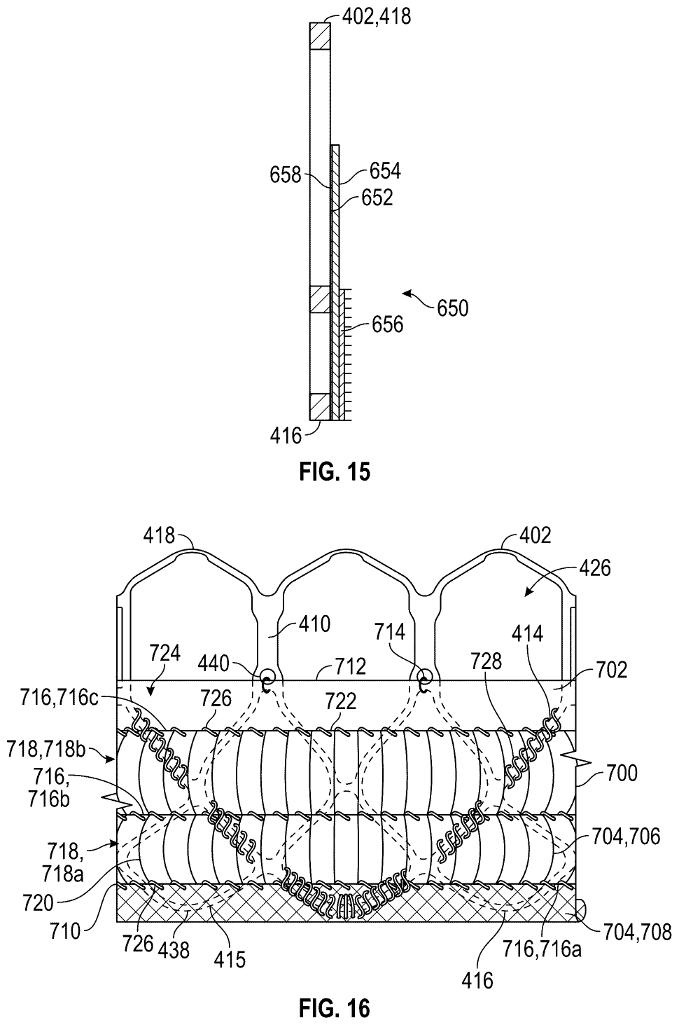

In some instances, the simulated micro scale structure can be formed on a surface of one of the layers or portions of the outer skirts described herein. As one example, as shown in FIG. 15, an outer skirt 650 can comprise an inner layer 652 comprising simulated micro scales, an intermediate layer 654 that can comprise a polymeric material (e.g., the same or similar to the second portion 504 described above), and an outer layer 656 the comprises a fabric (e.g., the same or similar to the first portion 502 described above). In some instances, the intermediate layer 654 extends beyond the outer layer 656, toward the outflow end 418 of the frame 402 and the inner layer 652 can cover all or a majority of an inner surface 658 of the intermediate layer 654. In other instances, the inner layer 652 can be part of (formed as one piece with) the intermediate layer 654 such that the inner layer 652 is an inner surface formed on the intermediate layer 654 (e.g., the material of the intermediate layer 654 is used in the negative mold 616 of the shark skin described above, thereby forming a material layer with one relatively smooth surface and an opposite surface comprising the simulated micro scales).

In other instances, the simulated micro scale structure can be applied to an alternate skirt as an inner layer comprising the simulated micro scales. For example, a more traditional fabric skirt, such as the outer skirt of FIG. 1 or FIG. 3 can include an inner layer, coating, or covering comprising the simulated micro scales that is adhered to an inner surface of the skirt fabric. As a result, the simulated micro scales can face the frame and the leaflets of the prosthetic heart valve.

By forming an outer skirt with an inner surface, coating or layer comprising the simulated micro scales (e.g., simulated shark skin surface), accumulation of debris and/or calcification on the inner surface of the outer skirt can be reduced or avoided, thereby increasing a long term durability and longevity of the prosthetic heart valve.

FIGS. 16-21 show additional examples of outer skirts for a prosthetic heart valve that can comprise a polymeric inner layer and a fabric outer layer that is attached to the inner layer. In some instances, the outer layer is attached to the inner layer via fasteners (such as sutures). The polymer inner layer can comprise a polymeric material (e.g., similar to the second portion 504 of the outer skirt 500, as described above with reference to FIGS. 10-12B) and can extend further toward an outflow end of the frame than the fabric outer layer. In some instances, the polymeric inner layer can extend over a portion of outflow cells of the frame (e.g., elongated outflow cells disposed at an outflow end of the frame). Further, in some instances, the fabric outer layer can comprise two or more fabric portions that are removably attached to the polymeric inner layer by a pull suture. As a result, the prosthetic heart valve to which the outer skirt is attached can effectively seal with the native anatomy at the implantation site, while also being more easily removed (explanted) from the native anatomy.

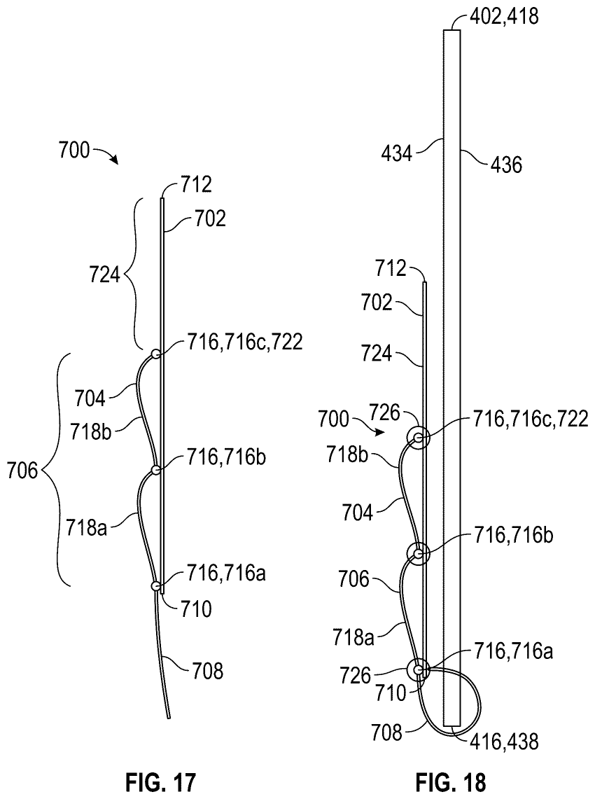

FIGS. 16-18 show a first example of such an outer skirt 700 that can be secured around an outer surface of the exemplary frame 402 (however, in alternate examples the outer skirt 700 can be secured around a differently configured frame, such as one of those referenced above). FIG. 16 shows a side view of portion of the outer skirt 700 secured to a portion of the frame 402 and FIGS. 17 and 18 show schematic, cross-sectional side views of the outer skirt 700 before (FIG. 17) and after securing to the frame 402 (FIG. 18).

The outer skirt 700 can comprise an inner layer 702 and an outer layer 704 attached to the inner layer 702. The inner layer 702 can comprise a polymeric material and the outer layer 704 can comprise a fabric. In some examples, the outer layer 704 can comprise two outer layers or outer layer portions, including a first outer layer portion 706 (also referred to as an upper or outflow outer layer portion) and a second outer layer portion 708 (also referred to as a lower or inflow outer layer portion). Further, in some instances, the outer layer 704 can extend axially beyond an outflow edge portion 722 of the first outer layer portion 706 toward the outflow end 418 of the frame 402.

The inner layer 702 can comprise a polymeric material, such as one of the materials described herein with reference to the second portion 504 of outer skirt 500 (e.g., TPU, ePTFE, or the like). In some instances, the inner layer 702 can be formed as a relatively thin layer such that the inner layer 702 does not increase a crimp profile of the prosthetic heart valve. For example, the inner layer 702 can have a thickness in a range of 5 to 20 μm.

In some instances, the inner layer 702 can comprise a sheet of polymeric material (e.g., TPU). In some instances, the inner layer 702 can include reinforcement members embedded within the polymeric material that are configured to increase is strength and resistance to tearing, such as weft and warp yarns (e.g., arranged at a non-zero angle, such as approximately 45 degrees, relative to a central longitudinal axis of the frame). Further, the polymeric material of the inner layer 702 can be configured such that if the leaflets of the prosthetic heart valve contact the inner layer 702, abrasion on the leaflets does not occur.

The inner layer 702 can extend from a location at or adjacent the inflow end 416 of the frame 402 toward the outflow end 418 of the frame 402 (FIGS. 16 and 18). In some instances, an inflow edge portion 710 of the inner layer 702 can extend over at least a portion of each inflow strut 415.

Additionally, in some examples, an outflow edge portion 712 of the inner layer 702 can extend over at least a portion (e.g., an inflow portion) of the elongated outflow cells 426 (FIG. 16). In some instances, as shown in FIG. 16, the outflow edge portion 712 can extend over be secured to inflow end portions of the axial struts 410 forming the outflow cells 426, such as to apertures 440 in the axial struts 410 via fasteners 714 (e.g., sutures). As described above, the polymeric material of the inner layer 702 can be elastic and configured to stretch (circumferentially) when the frame is radially expanded. As a result, the polymeric material of the inner layer 702 can form a snug fit around the outer surface of the frame 402 when the frame is radially expanded such that it lies flat against the frame with no excess slack that bulges outwardly or inwardly into the frame (e.g., through the outflow cells 426).

In alternate examples, the outer skirt 700 can be used with a differently configured frame, such as frame 12 of FIG. 1, frame 202 of FIG. 3, or any of various frames that are balloon-expandable, self-expandable or mechanically expandable, such as disclosed in the applications referenced above. Thus, in some instances, the outflow edge portion 712 of the inner layer 702 may not extend over outflow cells of the frame (e.g., the cells disposed in a row of cells defining the outflow end of the frame) and can instead extend to an intermediate portion of the frame, such as shown in FIGS. 1 and 3. However, in such instances, the inner layer 702 can still extend beyond the outer layer 704 toward the outflow end of the frame, as described further below.

The second outer layer portion 708 can comprise a woven fabric, such as a PET fabric. In alternate examples, the woven material of the second outer layer portion 708 can be another type of woven fabric. In some instances, the second outer layer portion 708 can include weft and warp yarns woven together in a plain weave. In some examples, the weft and warp yarns can be angled at 45 degrees (relative to the axial direction), as depicted schematically in FIG. 16. In some instances, the second outer layer portion 708 can comprise a woven fabric without any pile or floating fibers or yarns.

The second outer layer portion 708 can be secured to the inner layer 702 and extend toward the inflow end 416 of the frame 402 from the first outer layer portion 706 and beyond the inner layer 702 (FIGS. 16 and 18). Thus, the second outer layer portion 708 can be axially offset from the inflow edge portion 710 of the inner layer 702 (FIG. 17).

The second outer layer portion 708 can cover at least a portion of the inflow struts 415 (FIG. 16) and can wrap around inflow apices 438 at the inflow end 416 of the frame (FIGS. 16 and 18). In this way, the second outer layer portion 708 can wrap from the outer surface 434 of the frame 402, around the inflow apices 438, and across an inner surface 436 of the frame (inner surfaces of the inflow struts 415) (FIG. 18). In some examples, an end portion of the second outer layer portion 708 that wraps around the inflow apices 438 and to the inner surface 436 of the frame 402 can be secured to the inflow edge portion of the first outer layer portion 706 and the inflow edge portion 710 of the inner layer 702 (e.g., by stitches 726, as shown in FIG. 18).

By wrapping the second outer layer portion 708 around the inflow apices 438, such that the inflow apices 438 are covered by the second outer layer portion 708, the apices 438 can be prevented from rubbing against an inner surface of a guide sheath (or guide catheter) through which the prosthetic heart valve is advanced on the delivery apparatus toward the implantation site. As a result, push forces felt by a user advancing the delivery apparatus and prosthetic heart valve through the guide sheath can be reduced.

The first outer layer portion 706 can be a floating yarn or fiber portion comprising a plurality of circumferentially extending floating leno lines 716 (or woven portions) that are spaced axially apart from one another and a plurality of floating yarn sections 718 disposed between adjacent floating leno lines 716 (FIGS. 16-18). Each floating yarn section 718 can comprise a plurality of floating yarns 720 (or fibers) extending axially between and woven into adjacent leno lines 716. The floating yarns 720 can extend radially outward between the leno lines 716, thereby forming a textured outer surface on the first outer layer portion 706 that can be configured to increase tissue ingrowth following implantation of the prosthetic heart valve.

As depicted in FIGS. 16-18, the first outer layer portion 706 can include a first leno line 716a disposed adjacent the second outer layer portion 708, a second leno line 716b disposed at an intermediate portion of the first outer layer portion 706, and a third leno line 716c defining an outflow edge portion 722 of the first outer layer portion 706. Further, the first outer layer portion 706 can include a first floating yarn section 718a extending axially between the first leno line 716a and the second leno line 716b and a second floating yarn section 718b extending axially between the second leno line 716b and the third leno line 716c.

While three floating leno lines 176a, 716b, 716c are illustrated in the example of FIGS. 16-18, in other instances, the first outer layer portion 706 can include two floating leno lines 716 (e.g., at the outflow and inflow edge portions of the first outer layer portion 706, thereby defining a single floating yarn section 718 therebetween), or more than three floating leno lines (e.g., four, five, or the like), and thus more than two floating yarns sections 718.

The above-described configuration of the first outer layer portion 706, combined with the inner layer 702, allows for a number or density of the floating yarns 720 to be reduced (e.g., compared to a skirt not having an inner polymeric layer), thereby reducing a crimp profile of the prosthetic heart valve.

In some examples, in addition to the floating yarns 720 (which can be textured and extend outwardly, as described above), the floating yarn sections 718 can include one or more shorter yarns or cords that are shorter than the floating yarns 720 and dimensioned to limit a maximal axial length of the outer skirt 700 when the prosthetic heart valve is in a radially compressed configuration. For example, it may be desirable to limit the elongation of the outer skirt 700 when radially compressing the prosthetic heart valve to an amount that is similar to that of the frame. Thus, the additional shorter yarns can be dimensioned to match an elongation of the frame that occurs during radially compressing the prosthetic heart valve.

As shown in FIGS. 16-18, the outflow edge portion 722 of the first outer layer portion 706 is axially offset from the outflow edge portion 712 of the inner layer 702, thereby defining an outflow portion 724 of the inner layer 702 that is devoid of yarns. Thus, the outflow portion 724 can be configured to reduce perivalvular leakage across or through the frame 402 (for example, over upper regions of the scalloped line), while the first outer layer portion 706 can be configured to reduce or prevent perivalvular leakage axially along the prosthetic heart valve, between the frame and the native tissue.

In some examples, the first outer layer portion 706 and the second outer layer portion 708 of the outer layer 704 can be secured to the inner layer 702 by a plurality of fasteners, such as sutures. In some instances, as shown in FIG. 16, the first outer layer portion 706 and the second outer layer portion 708 of the outer layer 704 can be secured to the inner layer 702 by a plurality of whip stitches 726 that extend around the floating leno lines 716 (FIGS. 16 and 19).

The outer skirt 700 can then be secured to the frame 402 (or an alternate frame, as described above) by whip stitches, in-and-out stitches, or the like, that extend along a portion of the prosthetic heart valve that follows a scallop line of the leaflets and/or the outflow edge portion 712 of the outer skirt 700. As one example, as shown in FIG. 16, the outer skirt 700 can be secured to struts 414 of the frame 402 that extend along the scallop line of the leaflets by whip stitches 728 and to the apertures 440 in the axial struts 410 by fasteners 714 (e.g., sutures). In alternate examples, the outer skirt 700 can be secured at its inflow edge portion to the inflow struts 415 and at its outflow edge portion to the axial struts 410 (or alternate struts that the outflow edge portion 712 extends across).

In some examples, after implanting a prosthetic heart valve within the native tissue at an implantation site (e.g., in a native valve annulus), the prosthetic heart valve may need to be explanted (removed from the implantation site). However, since fabric skirts of prosthetic heart valve can be configured to encourage tissue ingrowth, it can be difficult to release the fabric skirts from the tissue and explant the prosthetic heart valve.

Thus, the inventors herein have realized that it is advantageous to provide a prosthetic heart valve with a fabric outer skirt that reduces perivalvular leakage, but can also be removably coupled to the frame of the prosthetic heart valve, thereby increasing an ease of explanation of the prosthetic heart valve.

FIG. 19 shows another example of an outer skirt 800 that can be secured around an outer surface of the exemplary frame 402. Similar to the outer skirt 700 of FIGS. 16-18, the outer skirt 800 can comprise an inner layer 802 (which can be similar to the inner layer 702 described above) and an outer layer 804 attached to the inner layer 802. The inner layer 802 can comprise a polymeric material and the outer layer 804 can comprise a fabric. As shown in FIG. 19, the outer layer 804 can include only the first outer layer portion 706, as described above with reference to FIGS. 16-19, and the inner layer 802 extends over and wraps around the inflow apices 438 of the frame 402. However, in alternate examples, the inner layer 802 can extend over the inflow struts 415 (e.g., as shown in FIG. 16), but may not wrap around the inflow apices 438.

The outer layer 804 can be attached to the inner layer 802 similarly to the outer skirt 700, as described above with reference to FIG. 16. However, the outer layer 804 can be releasably attached to the inner layer 802 by a plurality of whip stitches 812 and a pull suture 810. As a result, during an explanation procedure, the outer layer 804 can be released from the inner layer 802 (and the rest of the prosthetic heart valve, including the frame 402), and thus the prosthetic heart valve can be more easily removed from the implantation site (since the outer layer 804 may have tissue adhering or growing thereto).

As shown in FIG. 19, the pull suture 810 can extend along an upper edge portion 806 of the outer layer 804, and in some instances, along the third leno line 716c (FIG. 19). As shown in more detail in the schematic cross-sectional view of FIG. 20, the whip stitches 812 can extend around the pull suture 810 and through the outer layer 804 and the inner layer 802.

For example, as shown in FIG. 20, an inner surface 814 of the outer layer 804 contacts or is disposed against an outer surface 816 of the inner layer 802, and the inner layer 802 is disposed adjacent or against the outer surface 434 of the struts 414 of the frame 402. The pull suture 810 contacts or is disposed against an outer surface 818 of the outer layer 804.

The whip stitches 812 of a suture 820 can extend through the material of the inner layer 802, through the material of the outer layer 804 (e.g., passing through and being disposed within a plurality of apertures 822 in the material of the outer layer 804), and around the pull suture 810 in a whip stitch pattern along the length of the upper edge portion 806 of the outer layer 804. In some instances, the pull suture 810 can pass through and be disposed within a plurality of pre-formed apertures 824 in the inner layer 802.

Each whip stitch 812 includes a leading end portion 813a that extends through the material of the inner layer 802, or alternatively through an aperture 824 in the inner layer 802, outwardly through an aperture 822 in the outer layer 804, around the pull suture 810, and transitions to a trailing end portion 813b that extends inwardly through the same aperture 822 in the outer layer 804, and through a different aperture 824 in the inner layer 802 (or through the material of the inner layer 802), where the trailing end portion 813b transitions to the leading end portion of the next whip stitch 812 (FIG. 20). In this manner, each whip stitch 812 is threaded through a single opening or aperture 822 in the outer layer 804 and two different, but adjacent, apertures 824 in the inner layer 802. As can be seen in FIG. 20, the pull suture 810 prevents the whip stitches 812 from being pulled through the apertures 822 when the pull suture 810 is positioned along the stitch line across the upper edge portion 806. Therefore, the outer layer 804 can be secured to the inner layer 802, which is secured to the frame 402 (FIG. 19), via a force exerted by the whip stitches 812 on the pull suture 810 and the inner surface 826 of the inner layer 802 when the pull suture is disposed through the stitch line and across the upper edge portion 806.

In some examples, the apertures 822 in the material of the outer layer 804 and/or the apertures 824 in the material of the inner layer 802 can be pre-formed, such as by laser drilling, cutting, stamping or other suitable techniques known in the art. In other examples, the apertures 822 in the material of the outer layer 804 and/or the apertures 824 in the material of the inner layer 802 can be formed as the whip stitches 812 are stitched through the material of the outer layer 804 and/or the material of the inner layer 802.

When the pull suture 810 is removed (e.g., via pulling on one of the loose ends 828a or 828b (FIG. 20) in a direction parallel to the stitch line or outwardly from the frame 402), the whip stitches 812 are no longer retained by the pull suture 810. Thus, as the outer layer 804 is pulled away from the inner layer 802 and the frame 402 (or the inner layer 802 and the frame 402 are pulled away from the outer layer 804), the whip stitches 812 can be withdrawn through the apertures 822 of the outer layer 804, thereby resulting in the outer layer 804 separating (or detaching or uncoupling) from the inner layer 802 and the whip stitches 812 remaining attached to the inner layer 802.

Since the inner layer 802 can comprise a thromboresistant polymer (or a polymeric material having a greater thromboresistance than the fabric material of the outer layer 804), the inner layer 802 can resist tissue ingrowth and allow the inner layer 802 to be more easily separated from the outer layer 804 and the tissue at the implantation site.

In some instances, the suture 820 of the whip stitches 812 can be a thinner fiber, yarn, or suture relative to the thicker pull suture 810. In some instances, the suture 820 forming the whip stitches 812 can be comprised of a thinner, high tensile strength biocompatible material, such as an ultra-high molecular weight polyethylene (UHMPE) force Fiber® or other similar material or combinations thereof. In one specific implementation, the suture 820 can be formed from UHMPE force fiber and can be sufficiently thin and resistant to force such that the whip stitches 812 can cut through overgrown tissue on the outer surface of the outer layer 804 as the inner layer 802 is separated from the outer layer 804. In some instances, the pull suture 810 can be comprised of a thicker, high tensile strength, biocompatible material, such as a monofilament comprised of polypropylene (e.g., Prolene 4-0), polytetrafluoroethylene (PTFE), expanded PTFE (ePTFE), or other similar materials or combinations thereof. In one specific implementation, the pull suture 810 can be a microfilament comprised of Prolene, which is configured to readily separate from or be pulled away from any surrounding ingrown tissue when pulled on by the surgeon during an explant procedure.

In some examples, the pull suture 810 can have a distinct coloration or a colored coating (e.g., a green, black, or blue coloration or coating), can be radiopaque, and/or can be otherwise tagged such that it can be identified by a surgeon during an explant procedure. In some instances, an entire length of the pull suture 810 can, for example, be comprised of a brightly colored material or have a brightly colored coating. In other instances, a portion of the pull suture 810 (e.g., a free or loose end portion 828a/828b of the pull suture 810) can be comprised of a colored material, have a brightly colored coating, and/or can include a brightly colored tag or flap attached thereto. In other instances, the pull suture 810 or a portion thereof can include a material, coating, or tag that can be visualized using a specialized visualization apparatus. For example, the pull suture 810 can comprise a radiopaque material or one or more radiopaque markers that can be visualized via a fluoroscopy or x-ray device. For example, the one or more radiopaque markers can be embedded within or attached to an outer surface of the pull suture 810.

In some examples, the loose ends 828a, 828b of the pull suture 810 can be tied to a commissure of the prosthetic heart valve (e.g., commissure 856 shown in FIG. 21). As such, during explanation of the prosthetic heart valve, the loose end 828a or 828b of the pull suture 810 can easily and quickly be located by and surgeon and pulled, thereby pulling the pull suture 810 away from the outer skirt 800 and detaching the outer layer 804 from the inner layer 802.

FIG. 21 shows a prosthetic heart valve 850 comprising the frame 402, the outer skirt 800 disposed around and secured to an outer surface of the frame 402, and a valvular structure 852 comprising a plurality of leaflets 854 disposed within an interior of the frame. Commissure tabs of adjacent leaflets 854 are secured together within commissure windows of the frame 402, thereby forming commissures 856. As depicted in FIG. 21, the floating yarns 720 of the first outer layer portion 706 of the outer layer 804 of the outer skirt 800 can form a fuzzy, plush, or texturized outer surface 858 that is configured to contact the native tissue when the prosthetic heart valve 850 is radially expanded and implanted at an implantation site. As described herein, this texturized outer surface 858 can be configured to promote tissue ingrowth therein, thereby increasing perivalvular leakage sealing against the native tissue. At the same time, the prosthetic heart vale 850 can be more easily removed from the native tissue during an explanation procedure, due to the outer layer 804 being removably coupled to the inner layer 802, as described above with reference to FIGS. 19 and 20.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame having an inflow end, an outflow end, and an intermediate portion disposed between the inflow end and outflow end; and an outer skirt disposed around an outer surface of the frame, the outer skirt comprising: a first portion comprising a polymeric material; and a second portion comprising a fabric, wherein the polymeric material has a greater thromboresistance than the fabric, wherein the second portion is secured to the inflow end of the frame and extends toward the intermediate portion, and wherein the first portion extends from the second portion and toward the outflow end of the frame.

Example 2. The prosthetic heart valve of any example herein, particularly example 1, wherein the frame comprises a plurality of interconnected struts defining a plurality of circumferentially extending rows of cells including a first row of outflow cells disposed at the outflow end, wherein the outflow cells of the first row of outflow cells are elongated in an axial direction relative to remaining rows of cells of the plurality of rows of cells, and wherein the first portion of the outer skirt covers a portion of each outflow cell of the first row of outflow cells.

Example 3. The prosthetic heart valve of any example herein, particularly example 2, wherein the portion of the outflow cells is an inflow half of the outflow cells that is disposed closer to the inflow end than the outflow end of the frame.

Example 4. The prosthetic heart valve of any example herein, particularly either example 2 or example 3, wherein an interface between the first portion and the second portion of the outer skirt extends circumferentially around the frame at an axial position that is adjacent to inflow apices of the outflow cells.

Example 5. The prosthetic heart valve of any example herein, particularly any one of examples 2-3, wherein the plurality of circumferentially extending rows of cells further includes a second row of intermediate cells and a third row of inflow cells, and wherein the second portion extends across the second row of intermediate cells and the third row of inflow cells.

Example 6. The prosthetic heart valve of any example herein, particularly any one of examples 1-5, wherein the polymeric material is elastic and is configured to stretch circumferentially when the frame is radially expanded from a radially compressed configuration to a radially expanded configuration.

Example 7. The prosthetic heart valve of any example herein, particularly any one of examples 1-6, wherein the polymeric material is configured to form a snug fit around the outer surface of the frame when the frame is in a radially expanded configuration.

Example 8. The prosthetic heart valve of any example herein, particularly any one of examples 1-7, wherein the polymeric material is a non-textile.

Example 9. The prosthetic heart valve of any example herein, particularly any one of examples 1-8, wherein the polymeric material is thermoplastic polyurethane.

Example 10. The prosthetic heart valve of any example herein, particularly any one of examples 1-9, wherein the fabric of the second portion is a woven fabric comprising polyethylene terephthalate.

Example 11. The prosthetic heart valve of any example herein, particularly any one of examples 1-10, wherein the fabric of the second portion is a reinforced fabric comprising weft and warp fibers that are angled at a non-zero angle relative to a central longitudinal axis of the prosthetic heart valve.

Example 12. The prosthetic heart valve of any example herein, particularly any one of examples 1-11, wherein the fabric of the second portion comprises a plurality of radially outwardly extending fibers that is configured to contact and seal against a tissue.

Example 13. The prosthetic heart valve of any example herein, particularly any one of examples 1-12, wherein the first portion further extends across an inner surface of the second portion such that the first portion forms an inner layer of the outer skirt that is disposed against the outer surface of the frame and the second portion forms an outer layer of the outer skirt, the inner layer extending beyond the outer layer in an axial direction toward the outflow end of the frame.

Example 14. The prosthetic heart valve of any example herein, particularly example 13, wherein the first portion further extends across an outer surface of the second portion and encapsulates the second portion therein.

Example 15. The prosthetic heart valve of any example herein, particularly example 13, wherein the second portion is releasably attached to the first portion by a plurality of whip stitches and a pull suture, wherein the plurality of whip stitches extends around the pull suture and through the second portion and the first portion, and wherein the pull suture is configured to be pulled through and away from the whip stitches, thereby detaching the second portion from the first portion.

Example 16. The prosthetic heart valve of any example herein, particularly any one of examples 1-15, wherein the outer skirt further comprises a layer comprising a plurality of micro scales formed on a surface thereof disposed across an inner surface of one or more of the first portion and the second portion.

Example 17. The prosthetic heart valve of any example herein, particularly any one of examples 1-16, further comprising a valvular structure disposed in the frame and configured to regulate the flow of blood through the frame in one direction.

Example 18. A prosthetic heart valve comprising: an annular frame; and an outer skirt disposed around an outer surface of the frame, the outer skirt comprising: an outer layer comprising a fabric and forming an exposed surface for contacting a tissue; and an inner layer comprising a polymeric material and covering an inner surface of the outer layer, wherein the inner layer extends beyond an outflow edge portion of the outer layer toward an outflow end of the frame.

Example 19. The prosthetic heart valve of any example herein, particularly example 18, wherein an inflow edge portion of the outer layer is disposed at an inflow end of the frame.

Example 20. The prosthetic heart valve of any example herein, particularly either example 18 or example 19, wherein the frame comprises a plurality of interconnected struts defining a plurality of circumferentially extending rows of cells including a first row of outflow cells disposed at the outflow end of the frame, wherein the first row of outflow cells are longer in an axial direction relative to remaining rows of cells of the plurality of rows of cells, and wherein the inner layer of the outer skirt covers at least a portion of each outflow cell of the row of outflow cells.

Example 21. The prosthetic heart valve of any example herein, particularly example 20, wherein an outflow edge portion of the inner layer is secured to inflow end portions of axial struts of the frame that define the first row of outflow cells.

Example 22. The prosthetic heart valve of any example herein, particularly any one of examples 18-21, wherein the polymeric material is elastic and is configured to stretch circumferentially when the frame is radially expanded from a radially compressed configuration to a radially expanded configuration.

Example 23. The prosthetic heart valve of any example herein, particularly any one of examples 18-22, wherein the polymeric material has a greater thromboresistance than the fabric and is configured to form a snug fit around the outer surface of the frame when the frame is in a radially expanded configuration.

Example 24. The prosthetic heart valve of any example herein, particularly any one of examples 18-23, wherein the polymeric material is thermoplastic polyurethane.

Example 25. The prosthetic heart valve of any example herein, particularly any one of examples 18-24, wherein the outer layer comprises a first outer layer portion comprising a plurality of floating yarns that extend radially outward and away from the outer skirt and the frame.

Example 26. The prosthetic heart valve of any example herein, particularly example 25, wherein the first outer layer portion comprises a plurality of circumferentially extending floating leno lines that are spaced axially apart from one another and a plurality of floating yarn sections disposed between adjacent floating leno lines, each floating yarn section comprising floating yarns of the plurality of floating yarns that extend axially between and are woven into adjacent leno lines.

Example 27. The prosthetic heart valve of any example herein, particularly either example 25 or example 26, wherein an outflow edge portion of the first outer layer portion is attached to the inner layer by a plurality of whip stitches extending through the inner layer and the first outer layer portion.

Example 28. The prosthetic heart valve of any example herein, particularly example 27, wherein a pull suture extends along an outer surface of the outflow edge portion of the first outer layer portion, and wherein the first outer layer portion is releasably attached to the inner layer by the pull suture and the plurality of whip stitches which further extend around the pull suture.

Example 29. The prosthetic heart valve of any example herein, particularly any one of examples 25-28, wherein the outer layer further comprises a second outer layer portion that is secured to an inflow edge portion of the first outer layer portion and extends from the first outer layer portion toward an inflow end of the frame.

Example 30. The prosthetic heart valve of any example herein, particularly example 29, wherein the second outer layer portion extends beyond the inner layer toward the inflow end of the frame and wraps around inflow apices at the inflow end of the frame.

Example 31. The prosthetic heart valve of any example herein, particularly example 30, wherein an end portion of the second outer layer portion that wraps around the inflow apices is secured to the inflow edge portion of the first outer layer portion and an inflow edge portion of the inner layer.

Example 32. The prosthetic heart valve of any example herein, particularly any one of examples 29-31, wherein the second outer layer portion comprises a woven fabric.

Example 33. The prosthetic heart valve of any example herein, particularly any one of examples 18-12, further comprising a valvular structure disposed in the frame and configured to regulate the flow of blood through the frame in one direction.

Example 34. A prosthetic heart valve comprising: an annular frame having an inflow end, an outflow end, and an intermediate portion disposed between the inflow end and the outflow end; and an outer skirt disposed around an outer surface of the frame, the outer skirt comprising: an inner layer comprising a polymeric material; and an outer layer comprising a fabric and attached to the inner layer, the outer layer comprising a first outer layer portion and a second outer layer portion, wherein the first outer layer portion extends from the inflow end of the frame toward the intermediate portion of the frame, and wherein the second outer layer portion extends from the first outer layer portion toward the outflow end, and wherein an outflow edge portion of the inner layer extends axially beyond an outflow edge portion of the second outer layer portion toward the outflow end of the frame such that the outflow edge portion of the inner layer is disposed closer to the outflow end of the frame than the outflow edge portion of the second outer layer portion.

Example 35. The prosthetic heart valve of any example herein, particularly example 34, wherein the frame comprises a plurality of interconnected struts defining a plurality of circumferentially extending rows of cells including a first row of outflow cells disposed at the outflow end of the frame, wherein the first row of outflow cells are longer in an axial direction relative to remaining rows of cells of the plurality of rows of cells, and wherein the outer layer of the outer skirt covers at least a portion of each outflow cell of the row of outflow cells.

Example 36. The prosthetic heart valve of any example herein, particularly example 35, wherein the outflow edge portion of the inner layer is secured to apertures in inflow end portions of axial struts of the frame that define the first row of outflow cells.

Example 37. The prosthetic heart valve of any example herein, particularly either example 35 or example 36, wherein the frame is radially expandable between a radially compressed configuration and a radially expanded configuration, and wherein the polymeric material is elastic and is configured to stretch circumferentially when the frame is radially expanded from the radially compressed configuration to the radially expanded configuration.

Example 38. The prosthetic heart valve of any example herein, particularly any one of examples 34-37, wherein the polymeric material is configured to lie flat against the outer surface of the frame, without excess slack that bulges outwardly or inwardly into the frame, when the frame is in a radially expanded configuration.

Example 39. The prosthetic heart valve of any example herein, particularly any one of examples 34-38, wherein the polymeric material has a greater thromboresistance than the fabric.

Example 40. The prosthetic heart valve of any example herein, particularly any one of examples 34-39, wherein the polymeric material is thermoplastic polyurethane.

Example 41. The prosthetic heart valve of any example herein, particularly any one of examples 34-40, wherein the first outer layer portion comprises a woven fabric.

Example 42. The prosthetic heart valve of any example herein, particularly any one of examples 34-41, wherein the first outer layer portion extends axially beyond an inflow edge portion of the inner layer toward the inflow end of the frame and wraps around inflow apices at the inflow end of the frame.

Example 43. The prosthetic heart valve of any example herein, particularly example 42, wherein an end portion of the first outer layer portion that wraps around the inflow apices and over an inner surface of the frame is secured to an inflow edge portion of the second outer layer portion and an inflow edge portion of the inner layer.

Example 44. The prosthetic heart valve of any example herein, particularly any one of examples 34-43, wherein the second outer layer portion comprises a plurality of floating yarns that extend radially outward and away from the frame.

Example 45. The prosthetic heart valve of any example herein, particularly example 44, wherein the second outer layer portion comprises a plurality of circumferentially extending floating leno lines that are spaced axially apart from one another and a plurality of floating yarn sections disposed between adjacent floating leno lines, each floating yarn section comprising floating yarns of the plurality of floating yarns that extend axially between and are woven into adjacent leno lines.

Example 46. The prosthetic heart valve of any example herein, particularly any one of examples 34-45, wherein the outflow edge portion of the second outer layer portion is attached to the inner layer by a plurality of stitches extending through the inner layer and the second outer layer portion.

Example 47. The prosthetic heart valve of any example herein, particularly example 46, wherein a pull suture extends along an outer surface of the outflow edge portion of the second outer layer portion, and wherein the second outer layer portion is releasably attached to the inner layer by the pull suture and the plurality of stitches which further extend around the pull suture.

Example 48. The prosthetic heart valve of any example herein, particularly any one of examples 34-47, further comprising a valvular structure disposed in the frame and configured to regulate the flow of blood through the frame in one direction.

Example 49. A prosthetic heart valve comprising: an annular frame having an inflow end and an outflow end; and an outer skirt disposed around an outer surface of the frame, the outer skirt comprising: an inner layer; and a fabric outer layer releasably attached to the inner layer by a plurality of whip stitches and a pull suture, wherein the plurality of whip stitches extends around the pull suture and through the inner layer and the outer layer, and wherein the pull suture is configured to be pulled through and away from the whip stitches, thereby detaching the outer layer from the inner layer.

Example 50. The prosthetic heart valve of any example herein, particularly example 49, wherein the pull suture extends along an outflow edge portion of the outer layer, on an outer surface of the outer layer.

Example 51. The prosthetic heart valve of any example herein, particularly either example 49 or example 50, wherein each whip stitch is threaded through a single aperture in the outer layer and two adjacent apertures in the inner layer.

Example 52. The prosthetic heart valve of any example herein, particularly any one of examples 49-51, wherein the inner layer comprises a polymeric material.

Example 53. The prosthetic heart valve of any example herein, particularly example 52, wherein the frame is radially expandable between a radially compressed configuration and a radially expanded configuration, and wherein the polymeric material is elastic and is configured to stretch circumferentially when the frame is radially expanded from the radially compressed configuration to the radially expanded configuration.

Example 54. The prosthetic heart valve of any example herein, particularly either example 52 or example 53, wherein the polymeric material is configured to lie flat against the outer surface of the frame, without excess slack that bulges outwardly or inwardly into the frame, when the frame is in a radially expanded configuration.

Example 55. The prosthetic heart valve of any example herein, particularly any one of examples 52-54, wherein the polymeric material has a greater thromboresistance than a fabric of the outer layer.

Example 56. The prosthetic heart valve of any example herein, particularly any one of examples 52-55, wherein the polymeric material is thermoplastic polyurethane.

Example 57. The prosthetic heart valve of any example herein, particularly any one of examples 52-56, wherein an outflow edge portion of the inner layer extends beyond an outflow edge portion of the outer layer toward the outflow end of the frame.

Example 58. The prosthetic heart valve of any example herein, particularly any one of examples 52-57, wherein the inner layer is secured to struts of the frame.

Example 59. The prosthetic heart valve of any example herein, particularly any one of examples 52-58, wherein an inflow edge portion of the inner layer extends around the inflow end of the frame.

Example 60. The prosthetic heart valve of any example herein, particularly any one of examples 52-59, wherein the outer layer comprises a plurality of floating yarns that extend radially outward and away from the frame.

Example 61. The prosthetic heart valve of any example herein, particularly example 60, wherein the outer layer comprises a plurality of circumferentially extending floating leno lines that are spaced axially apart from one another and a plurality of floating yarn sections disposed between adjacent floating leno lines, each floating yarn section comprising floating yarns of the plurality of floating yarns that extend axially between and are woven into adjacent leno lines.

Example 62. The prosthetic heart valve of any example herein, particularly any one of examples 52-61, further comprising a valvular structure disposed in the frame and configured to regulate the flow of blood through the frame in one direction.

Example 63. A prosthetic heart valve comprising: an annular frame; a valvular structure disposed in the frame and configured to regulate the flow of blood through the frame in one direction; and a skirt coupled to the frame, the skirt comprising at least one layer comprising a plurality of micro scales formed on a surface thereof.

Example 64. The prosthetic heart valve of any example herein, particularly example 63, wherein the plurality of micro scales is configured to reduce drag across the surface of the skirt.

Example 65. The prosthetic heart valve of any example herein, particularly either example 63 or example 64, wherein the skirt is an outer skirt disposed around an outer surface of the frame.

Example 66. The prosthetic heart valve of any example herein, particularly example 65, wherein the layer comprising the plurality of micro scales is an inner layer of the skirt that is disposed against the frame and the skirt further comprises an outer layer comprising a fabric.

Example 67. The prosthetic heart valve of any example herein, particularly either example 63 or example 64, wherein the skirt is an inner skirt disposed around an inner surface of the frame, between the valvular structure and the frame.

Example 68. The prosthetic heart valve of any example herein, particularly any one of examples 63-67, wherein the surface of the layer of the skirt including the micro scales formed thereon is a radially inwardly facing surface that faces the valvular structure.

Example 69. A prosthetic heart valve comprising: an annular frame; and an outer skirt disposed around an outer surface of the frame, the outer skirt comprising: a base layer comprising a polymeric material, the base layer disposed against the outer surface of the frame; and a plurality of outwardly extending yarns adhered to an outer surface of the base layer and extending radially outward and away from the base layer.

Example 70. The prosthetic heart valve of any example herein, particularly example 69, wherein the polymeric material has a greater thromboresistance that the plurality of outwardly extending yarns.

Example 71. The prosthetic heart valve of any example herein, particularly either example 69 or example 70, wherein the polymeric material is thermoplastic polyurethane.

Example 72. The prosthetic heart valve of any example herein, particularly any one of examples 69-71, wherein the yarns of the plurality of outwardly extending yarns are individual yarns that are spaced apart from one another across the outer surface of the base layer.

Example 73. The prosthetic heart valve of any example herein, particularly any one of examples 69-72, wherein the yarns of the plurality of outwardly extending yarns are adhered to the base layer with an adhesive.

Example 74. The prosthetic heart valve of any example herein, particularly any one of examples 69-72, wherein the yarns of the plurality of outwardly extending yarns are adhered to the base layer by chemical bonding.

Example 75. The prosthetic heart valve of any example herein, particularly any one of examples 69-74, wherein the plurality of outwardly extending yarns is disposed across an intermediate portion of the base layer, and wherein an outflow edge portion of the base layer that is devoid of outwardly extending yarns extends beyond the intermediate portion toward an outflow end of the frame.

Example 76. The prosthetic heart valve of any example herein, particularly example 75, wherein an inflow edge portion of the base layer that is devoid of outwardly extending yarns extends beyond the intermediate portion toward an inflow end of the frame.

Example 77. A method comprising sterilizing the prosthetic heart valve, apparatus, and/or assembly of any example.

Example 78. A prosthetic heart valve of any one of examples 1-76, wherein the prosthetic heart valve is sterilized.

The features described herein with regard to any example can be combined with other features described in any one or more of the other examples, unless otherwise stated. For example, any one or more of the features of one outer skirt for a prosthetic heart valve can be combined with any one or more features of another outer skirt for a prosthetic heart valve. As another example, any one or more features of one frame for a prosthetic heart valve can be combined with any one or more features of another frame for a prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame having an inflow end, an outflow end, and an intermediate portion disposed between the inflow end and outflow end; and
an outer skirt disposed around an outer surface of the frame, the outer skirt comprising:
a first portion comprising a polymeric material; and
a second portion comprising a fabric, wherein the polymeric material has a greater thromboresistance than the fabric,
wherein the second portion is secured to the inflow end of the frame and extends toward the intermediate portion, and wherein the first portion extends from the second portion and toward the outflow end of the frame.

2. The prosthetic heart valve of claim 1, wherein the frame comprises a plurality of interconnected struts defining a plurality of circumferentially extending rows of cells including a first row of outflow cells disposed at the outflow end, wherein the outflow cells of the first row of outflow cells are elongated in an axial direction relative to remaining rows of cells of the plurality of rows of cells, and wherein the first portion of the outer skirt covers a portion of each outflow cell of the first row of outflow cells.

3. The prosthetic heart valve of claim 2, wherein an interface between the first portion and the second portion of the outer skirt extends circumferentially around the frame at an axial position that is adjacent to inflow apices of the outflow cells.

4. The prosthetic heart valve of claim 1, wherein the polymeric material is elastic and is configured to stretch circumferentially when the frame is radially expanded from a radially compressed configuration to a radially expanded configuration.

5. The prosthetic heart valve of claim 1, wherein the polymeric material is a non-textile.

6. The prosthetic heart valve of claim 1, wherein the polymeric material is thermoplastic polyurethane.

7. The prosthetic heart valve of claim 1, wherein the fabric of the second portion is a woven fabric comprising polyethylene terephthalate.

8. The prosthetic heart valve of claim 1, wherein the fabric of the second portion comprises a plurality of radially outwardly extending fibers that is configured to contact and seal against a tissue.

9. The prosthetic heart valve of claim 1, wherein the first portion further extends across an inner surface of the second portion such that the first portion forms an inner layer of the outer skirt that is disposed against the outer surface of the frame and the second portion forms an outer layer of the outer skirt, the inner layer extending beyond the outer layer in an axial direction toward the outflow end of the frame.

10. A prosthetic heart valve comprising:
an annular frame; and
an outer skirt disposed around an outer surface of the frame, the outer skirt comprising:
an outer layer comprising a fabric and forming an exposed surface for contacting a tissue; and
an inner layer comprising a polymeric material and covering an inner surface of the outer layer, wherein the inner layer extends beyond an outflow edge portion of the outer layer toward an outflow end of the frame.

11. The prosthetic heart valve of claim 10, wherein an inflow edge portion of the outer layer is disposed at an inflow end of the frame.

12. The prosthetic heart valve of claim 10, wherein the frame comprises a plurality of interconnected struts defining a plurality of circumferentially extending rows of cells including a first row of outflow cells disposed at the outflow end of the frame, wherein the first row of outflow cells are longer in an axial direction relative to remaining rows of cells of the plurality of rows of cells, and wherein the inner layer of the outer skirt covers at least a portion of each outflow cell of the row of outflow cells.

13. The prosthetic heart valve of claim 10, wherein the polymeric material has a greater thromboresistance than the fabric and is configured to form a snug fit around the outer surface of the frame when the frame is in a radially expanded configuration.

14. The prosthetic heart valve of claim 10, wherein the outer layer comprises a first outer layer portion comprising a plurality of floating yarns that extend radially outward and away from the outer skirt and the frame.

15. The prosthetic heart valve of claim 14, wherein the outer layer further comprises a second outer layer portion that is secured to an inflow edge portion of the first outer layer portion and extends from the first outer layer portion toward an inflow end of the frame.

16. The prosthetic heart valve of claim 15, wherein the second outer layer portion extends beyond the inner layer toward the inflow end of the frame and wraps around inflow apices at the inflow end of the frame.

17. The prosthetic heart valve of claim 15, wherein the second outer layer portion comprises a woven fabric.

18. A prosthetic heart valve comprising:
an annular frame; and
an outer skirt disposed around an outer surface of the frame, the outer skirt comprising:
a base layer comprising a polymeric material, the base layer disposed against the outer surface of the frame; and
a plurality of outwardly extending yarns adhered to an outer surface of the base layer and extending radially outward and away from the base layer.

19. The prosthetic heart valve of claim 18, wherein the polymeric material has a greater thromboresistance that the plurality of outwardly extending yarns.

20. The prosthetic heart valve of claim 18, wherein the polymeric material is thermoplastic polyurethane.
